(19) United States
(12) Patent Application Publication
(10) Pub. No.: US 2002/0019350 A1
(43) Pub. Date: Feb. 14, 2002

(54) Targeted angiogenesis

(76) Inventors: Arnold J. Levine, New York, NY (US); Artur Mitterer, Orth, Donau (AT); Falko-Guenter Falkner, Orth, Donau (AT); Friedrich Scheiflinger, Vienna (AT); Friedrich Dorner, Vienna (AT)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/782,650

(22) Filed: Feb. 12, 2001

Related U.S. Application Data
(62) Division of application No. 09/327,045, filed on Jun. 7, 1999, now abandoned.

(51) Int. Cl. A61K 38/18

(52) U.S. Cl. 514/12; 530/399

(57) ABSTRACT

The invention relates to compositions, methods, and gene therapy reagents to promote or to inhibit angiogenesis in the treatment of peripheral vascular or cardiovascular diseases, utilizing a chimeric molecule comprising an angiogenic factor linked to a targeting molecule that specifically binds to a vascular endothelium.

### FIELD OF THE INVENTION

This invention relates to compositions, methods, and gene therapy reagents to promote or to inhibit angiogenesis in vivo for the treatment of peripheral vascular or cardiovascular diseases. In particular, this invention pertains to the use of an angiogenic factor linked to a targeting molecule that specifically binds to a vascular endothelium for inducing angiogenesis.

### BACKGROUND OF THE INVENTION

Angiogenesis is the process of developing new blood vessels that involves the proliferation, migration and tissue infiltration of capillary endothelial cells from pre-existing blood vessels. Angiogenesis is important in normal physiological processes including embryonic development, follicular growth, and wound healing as well as in pathological conditions involving tumor growth and non-neoplastic diseases involving abnormal neovascularization, including neovascular glaucoma (see, e.g., Folkman, J. et al., Science (1987) 235: 442-447).

Diseases and conditions causing or involving tissue ischemia are major health concerns. Ischemia is seen, for example, in coronary artery disease (CAD) and peripheral vascular disease (PVD). It has been reported by the American Heart Association that there are about 60 million adults in the United States with cardiovascular disease, including 11 million adults with coronary heart disease. Angina, a symptom of heart ischemia, afflicts 1.5 million adults in the United States, with about 350,000 new cases a year. It is estimated that PVD affects 30 percent of the adult population. A primary cause of PVD, atherosclerotic vascular disease, coronary heart disease (CHD), and cerebrovascular disease is diabetes mellitus.

Ischemia occurs when a tissue receives an inadequate supply of blood. For example, myocardial ischemia occurs when cardiac muscle does not receive an adequate blood supply. This can be due to occlusion or narrowing of the blood vessels, such as seen in coronary artery atherosclerosis. Treatments include surgical and pharmaceutical approaches. Surgical intervention is used to widen the narrowed lumens (e.g., balloon angioplasty) or to increase the numbers of cardiac blood vessels (e.g., bypass surgery using grafts). Less traumatic pharmaceutical treatments act to decrease cardiac muscle demand for oxygen and nutrients or to increase the blood supply. Oxygen demand can be lowered by decreasing the contractile response of the heart to a hemodynamic load (e.g., using beta-adrenergic blockers). Cardiac blood supply can be augmented by increasing the diameter of smooth muscle-walled coronary artery vessel lumens (as with nitroglycerin or calcium channel blockers). However, these pharmaceutical treatments are inexact, transiently active, and highly prone to drug interactions and side effects.

Another means to increase blood supply to an ischemic tissue is to induce the growth of blood vessels to the tissue through angiogenesis or to increase the amount of blood bathing the tissues referred to as increased blood perfusion. This can be accomplished by administration of angiogenic factors. Several factors have been implicated as possible regulators of angiogenesis in vivo. These include transforming growth factor (TGFB), acidic and basic fibroblast growth factor (aFGF and bFGF), platelet derived growth factor (PDGF), and vascular endothelial growth factor (VEGF) (see, e.g., Klagsbrun, M. et al., Annual Rev. Physiol. (1991) 53: 217-239). VEGF, an endothelial cell-specific mitogen, is distinct among these factors in that it acts as an angiogenesis inducer by specifically promoting the proliferation of endothelial cells.

VEGFs are important mediators of angiogenesis, as they act directly and specifically on endothelial cells. See, e.g., Grad et al., Clin. Chem Lab Med. (1998) 36: 379-383. In vivo, they are associated with blood vessel growth in development, wound repair (angiogenesis is a key component of the repair mechanisms triggered by tissue injury), cancer, and other diseases and conditions.

To achieve an angiogenic effect, repeated and/or long term administration of a polypeptide angiogenic factor, such as VEGF, would be needed. This approach, however, is typically very costly and inconvenient, as it usually requires repeated administrations by injection.

Alternatively, a polypeptide angiogenic factor can be administered in vivo by delivering not the polypeptide itself, but instead, the nucleic acid which encodes it. Angiogenic genes have been administered in vivo intravascularly. See, e.g., Laitinen, et al., Hum. Gene Ther. (1998) 9: 1481-1486; Isner, et al., Adv. Drug Deliv. Reviews (1997) 30: 185-197; Giordano et al., Nature Med. (1996) 2: 534-539; Takeshita, et al., Lab. Invest. (1996) 75: 487-501; Mc Donald, et al., U.S. Pat. No. 5,837,283 (the “'283” patent)

Polypeptide-encoding genes have been injected intramuscularly (as naked plasmid DNA or viral expression vectors). See, e.g., Baumgartner, I. et al., Circulation (1998) 97: 1114-1123; Tsurumi Y, et al. Circulation (1997) 96(9 Suppl): II-382-8; Takeshita, S. et al., Lab Invest (1996) 75: 487-501; Hammond, U.S. Pat. No. 5,792,453; and McDonald, the '283 patent (supra). See also Majesky, M. Circulation (1996) 94: 3062-4.

Despite recent advances in identifying genes encoding ligands and receptors involved in angiogenesis, there is no indication that the current methods would promote the level of angiogenesis required to overcome peripheral or cardiac ischemias. For example, in existing therapy, there is the need for repeated or long term delivery of the angiogenic proteins to achieve an angiogenic effect. This can limit the utility of using these proteins to stimulate angiogenesis in clinical settings. In other words, successful therapy in humans would require sustained and long-term infusion of one or more of these angiogenic peptides or proteins, which are themselves prohibitively expensive and which would need to be delivered by catheters placed in the coronary arteries, further increasing the expense and difficulty of treatment.

Considering the increasing numbers of individuals in our aging population afflicted with disease and conditions involving ischemic tissues, new treatments for ischemia that are safer, more predictable, and easier to administer are needed. The present invention provides these needs and related advantages.

### SUMMARY OF THE INVENTION

This invention provides a chimeric molecule comprising an angiogenic factor linked to a targeting molecule that specifically binds to a vascular endothelium. Some such chimeric molecules are fusion proteins, wherein the fusion proteins comprise an angiogenic factor linked to a targeting molecule that specifically binds to a vascular endothelium.

This invention also provides a method of inducing angiogenesis. This method comprises contacting a cell with a chimeric molecule wherein the chimeric molecule comprises an angiogenic factor attached to a targeting molecule that specifically binds to a vascular endothelium.

This invention further provides a method for increasing cardiac neovascularization. This method comprises contacting an endothelial cell of the cardiac vasculature with a chimeric molecule wherein the chimeric molecule further comprises an angiogenic factor linked to a targeting molecule that specifically binds to a vascular endothelium.

This invention further provides a method for increasing neovascularization in ischemic tissue in the peripheral vascular system.

This invention further provides a polynucleotide comprising a nucleic acid sequence encoding a fusion protein. The fusion protein further comprises an angiogenic factor and a targeting molecule, wherein the targeting molecule binds to a vascular endothelium.

This invention further provides a method of inducing angiogenesis in a tissue, the method comprises transfecting an endothelial cell with a nucleic acid wherein a fusion protein comprises an angiogenic factor and a targeting molecule, whereby the cell expresses a fusion protein encoded by the nucleic acid.

This invention further provides pharmaceutical compositions. The pharmaceutical compositions comprise a chimeric molecule wherein the chimeric molecule comprises an angiogenic factor linked to a targeting molecule that specifically binds to a vascular endothelium and a pharmaceutically acceptable carrier. Other pharmaceutical compositions comprise fusion proteins. The fusion proteins comprise an angiogenic factor and a targeting molecule, wherein the targeting molecule specifically binds to a vascular endothelium.

### DETAILED DESCRIPTION

### DEFINITIONS

The term “angiogenesis” refers to the process by which new blood vessels develop from preexisting vasculature, e.g., capillaries, see e.g., Folkman et al., Nature Med. (1992) 1: 27-21. Angiogenesis is a complex process (see Folkman et al., J Biol Chem. (1992) 267: 10931-4 and Fan et al., Trends Pharmacol Sci. (1995) 16: 57-66; these references and the references cited therein are incorporated herein by reference) that can involve endothelial cell and pericyte activation; basal lamina degradation; migration and proliferation (i.e., cell division) of endothelial cells and pericytes; formation of a new capillary vessel lumen; appearance of pericytes around the new vessels; development of a new basal lamina; capillary loop formation; persistence of involution, differentiation of the new vessels; and, capillary network formation and, eventually, organization into larger microvessels. See, e.g., Safi, J., et al., Mol. Cell Cardiol. (1997) 29: 2311-2325. Compositions can be screened for angiogenic activity in vitro or in vivo. An exemplary in vitro capillary formation assessment uses endothelial cells imbedded in Matrigel matrix (Collaborative Research, Bedford, Mass.), as described by, e.g., Deramaudt, et al., J. Cell. Biochem. (1998) 68: 121-127. In vivo animal models are discussed below. The term “vascular endothelium” means a thin layer of flat epithelial cells that lines, for example, serous cavities, lymph vessels, and blood vessels. The vascular endothelium plays important roles in the regulation of vascular tone, hemostasis, immune and inflammatory responses (see, e.g., Vane J., et al., New Engl. J. Med (1990) 323: 27-31; this reference and all references cited therein are incorporated herein by reference). These biological reactions can involve close interactions between circulating cells and the vascular endothelium. Adhesion of leukocytes to the vascular endothelium can be one of the most important events in the reaction to all forms of injury (see, e.g., Robert, S., et al., Am J Med Sci, (1994) 307: 378-389; Albelda, S. et al., FASEB J. (1994) 8: 504-512; Westlin, W. et al., Am J Pathol, (1993) 142: 1598-1609; these references and all references cited therein are incorporated herein by reference). Interaction of endothelial cells with activated leukocytes can be associated with defective endothelium-dependent vasodilation, increase in vascular permeability and in activation of the coagulation cascade. Many leukocyte products, including reactive oxygen species, superoxide and inflammatory cytokines, can impair endothelial function and can create the potential for a positive feedback loop between inflammation and coagulation. These molecules can play a role in a number of pathological processes including, but not limited to atherosclerosis, transplant rejection, septic shock, late phase hypersensitivity reactions and reperfusion injury (see, e.g., Carlos, T. et al., Blood (1994) 84: 2068-2101; Robert, S., et al., supra)

Angiogenesis is normally observed in wound healing, fetal and embryonal development and formation of the corpus luteum, endometrium and placenta. Persistent, unregulated angiogenesis occurs in a multiplicity of disease states, including but not limited to, tumor metastasis in cancer and abnormal growth by endothelial cells and supports the pathological damage seen in these conditions. The diverse pathological disease states in which unregulated angiogenesis can be present have been grouped together as “angiogenic-dependent” or “angiogenic-associated diseases.” Diseases and processes that are mediated by angiogenesis include, but are not limited to, hemangioma, solid tumors, blood borne tumors, leukemia, metastasis, psoriasis, scleroderma, phygenic granuloma, myocardial angiogenesis, Crohn's disease, plaque neovascularization, coronary collaterals, cerebral collaterals, arteriovenous malformations, ischemic limb angiogenesis, corneal diseases, neovascular glaucoma, diabetic retinopathy, arthritis, diabetic neovascularization, macular degeneration, wound healing, peptic ulcers, Helicobacter related diseases, and vasculogenesis.

The terms “angiogenic activity,” “angiogenic factor activity,” “vascular endothelial growth factor activity,” and “neovascularization” include a broad range of physiologic activities that increase the amount of blood flow to a tissue, including, e.g., increased vascular permeability, increased vascular density, endothelial cell (EC) activation, EC migration, EC proliferation, capillary formation (angiogenesis), vasculogenesis (the de novo organization of ECs into vascular structures); see, e.g., Folkman et al. (1992) supra). Angiogenic activity can include, e.g., angiogenic factors that induce angiogenesis, or angiogenic factors that inhibit angiogenesis, or angiogenic factors which induce expression of endogenous growth factors (e.g., gene activators or transcriptional regulators). The angiogenic factors include, but are not limited to, any protein, peptide, chemical molecule, or other molecule, which acts to induce or inhibit vascular growth. Angiogenic factors can be naturally or non-naturally occurring. A variety of methods can be used to determine the angiogenic activity of a given factor using biological activity assays such as the bovine capillary endothelial cell proliferation assay. Other bioassays include the chick CAM assay, the mouse corneal assay, and the effect of administering isolated or synthesized proteins on implanted tumors. The chick CAM assay is described by O'Reilly, et al. Cell, (1994) 79: 315-328. Many systems are available for assessing angiogenesis. For example, as angiogenesis is required for solid tumor growth, the inhibition of tumor growth in an animal model can be used as an index of the inhibition of angiogenesis. Angiogenesis can also be assessed in terms of models of wound-healing, in cutaneous or organ wound repair; and in chronic inflammation, e.g., in diseases such as rheumatoid arthritis, atherosclerosis and idiopathic pulmonary fibrosis (IPF). Angiogenic factor activity can also be assessed by counting vessels in tissue sections, e.g., following staining for marker molecules (e.g., CD3H, Factor VIII or PECAM-1). Other systems that can be used for assessing angiogenic factor activity include an endothelial cell chemotaxis assay. An angiogenic factor or agent can be identified in such an assay by its ability to promote endothelial cell chemotaxis above control values. Inhibition of endothelial cell chemotaxis can provide evidence of anti-angiogenic activity. Anti-angiogenic factors or agents can be identified by consistently reducing the endothelial cell chemotaxis back below the levels stimulated by an angiogenic agent.

The terms “vascular endothelial growth factor” or “VEGF” includes a family of growth factors which, alone or in combination with other growth factors, such as fibroblast growth factor (discussed below), can initiate vascular development, angiogenesis and other angiogenic activities (see, e.g., Claesson-Welsh, L. (ed.), Current Top. Microbiol. Immunol., Vol. 237 (Springer Publishing 1999); this reference and all references cited therein are incorporated herein by reference). The VEGF family includes VEGF (referred to as VEGF-A; see, e.g., Leung et al., Science (1989) 246: 1306-1309). The VEGF-A gene is organized in eight exons, separated by seven introns. Alternative exon splicing of a single VEGF-A gene results in the generation of four molecular species, encoding human proteins of 121, 165, 189, and 206 amino acids (VEGF-A₁₂₁, VEGF-A₁₆₅, VEGF-A₁₈₉, and VEGF-A₂₀₆; mouse VEGF-A isoforms have one amino acid less than the human isoforms); see, e.g., Carmeliet, P. et al., Am. J Physiol. (1997) 273(5, Part 2): H2091-104; U.S. Pat. Nos. 5,194,596; 5,240,848; and 5,332,671. Other family members include VEGF-B (see, e.g., Olofsson et al., Proc. Natl. Acad. Sci. USA (1996) 93: 2576-2581; this reference and all references cited therein are incorporated herein by reference); also referred to as “VRF”; see, e.g., Grimmond, S. et al., Genome Res. (1996) 6: 124-131); VEGF-C (see, e.g., Joukov, V. et al., EMBO J (1996) 15: 290-298 and WO96/39515; these references and all references cited therein are incorporated herein by reference); also referred to as VEGF-related protein or “VRP”; see, e.g., Lee, J. et al., Proc. Natl. Acad. Sci. USA (1996) 93: 1988-1992); VEGF-D (referred to as “FIGF”, see, e.g., Orlandini, M. et al., Proc. Natl. Acad. Sci. USA (1996) 93: 11675-11680 and Yamada, Y. et al., Genomics, (1997) 42: 483-488); and placenta growth factor (PlGF) (see, e.g., Maglione, D. et al., Proc. Natl. Acad. Sci. USA (1991) 88: 9267-9271). Recently, VEGF-E, a fifth VEGF family member, has been isolated and characterized (see, e.g., Ogawa, S. et al., J. Biol. Chem. (1998) 273(47): 31273-31282 and Meyer, M. et al., EMBO J (1999) 18(2): 363-374).

Human placenta growth factor (PlGF) is a glycosylated homodimer which shares 46% homology with VEGF at the protein level. Differential splicing of human PlGF mRNA can lead to either a 170 amino acid or 149 amino acid precursor, which are proteolytically processed to mature forms of 152 or 131 amino acids in length, respectively. See, e.g., Bayne and Thomas EP 0506477; Maglione, D. et al., Oncogene (1993) 8: 925-931; Hauser, S. and Weich, H., Growth Factors (1993) 9: 259-268; these references and references cited therein are incorporated herein by reference.

The terms “fibroblast growth factor” or “FGF” includes a family of growth factors which, alone or in combination with other growth factors, such as the VEGF family of growth factors, can initiate vascular development, angiogenesis and other angiogenic activities. The FGF family includes at least twenty polypeptides (see, e.g., Goncalves, L., Rev Port Cardiol (1998) 17 Suppl 2: II11-20; this reference and all references cited therein are incorporated herein by reference). Acidic FGF (aFGF or FGF-1) and basic FGF (bFGF or FGF-2) are the most extensively characterized members of this family. See, e.g., Klagsbrun, M, Prog Growth Factor Res (1989) 1: 207-35; Schelling, M., et al., Ann N YAcad Sci. (1991) 638:467-9; and Slavin, J., Cell Biol Int (1995) 19: 431-44; these references and all references cited therein are incorporated herein by reference.

Other angiogenic factors that induce angiogenesis include, but are not limited to the angiopoietin protein family (see, e.g., Davis, S., Curr Top Microbiol Immunol. (1999) 237:173-85; Papapetropoulos A. et al., Lab Invest (1999) 79: 213-23; Valenzuela, D., Proc Natl Acad Sci USA (1999) 96: 1904-9; Suri et al., Cell (1996) 87: 1171-1180; Takehara et al., Cell (1987) 49: 415-422; Suri et al., Cell (1996) 87: 1171-1180; these references and references cited therein are incorporated herein by reference). The term “angiopoietin-1” or “Ang1” refers to a protein that is a ligand for the Tie-2 receptor (see, e.g., Davis, S. et al., Science (1994) 266: 816-819). Ang1 can stimulate the Tie-2 receptor (as an agonist). The term “angiopoietin-2” or “Ang2” refers to a protein that can block Ang1-stimulated activation (as an antagonist) of the Tie-2 receptor (see, e.g., Maisonpierre, P. et al., Science (1997) 277: 55-60). The blocking of Ang1-stimulated activation can disrupt angiogenesis in vivo.

The term “homolog of VEGF” includes, but is not limited to, homodimers of VEGF-A, VEGF-B, VEGF-C, VEGF-D, VEGF-E and PIGF and any functional heterodimers formed between VEGF-A, VEGF-B, VEGF-C, VEGF-D, VEGF-E, including but not limited to a VEGF-A/VEGF-B heterodimer.

Communication between cells during vascular development and angiogenesis can involve at least five endothelial cell-specific tyrosine kinase receptors (see, e.g., Claesson-Welsh, L. (ed.), Current Top. Microbiol. Immunol., Vol. 237 (Springer Publishing 1999) and Mustonen, T. et al., J Cell. Biol. (1995) 129: 895-898; these references and all references cited therein are incorporated herein by reference) belonging to at least two distinct subclasses: two receptors of the Tie family (see, e.g., Partanen et al., Curr. Top. Microbiol Immunol (1999) 237: 158-171) and three VEGF receptors: VEGFR-1, -2, and -3. These three VEGF receptors were originally named Flt1 (Fms-like tyrosine kinase; see, e.g., De Vries, C. et al., Science (1992) 255: 989-991), KDR/Flk-1 (kinase insert-domain containing receptor or fetal-liver kinase-1; see, e.g., Terman et al., Biochem. Biophys. Res. Commun. (1992) 187: 1579-1586) and Flt4 (see, e.g., Pajusola, K. et al., Cancer Res (1992) 52: 5738-5743 and Galland, F. et al., Oncogene (1993) 8: 1233-1240) respectively. The biological response of VEGF is mediated through these high affinity VEGF receptors.

FGF receptors have also been characterized and include FGFR-1, FGFR-2, FGFR-3 and FGFR-4 (see, e.g., Klint, P. et al., Front Biosci. (1999) 15: D165-77 and Galzie, Z. et al., Biochem Cell Biol. (1997) 75: 669-85; these references and all references cited therein are incorporated herein by reference).

A variety of in vivo animal models can be used to evaluate the ability of chimeric molecules of the invention to have angiogenic activity (in addition to the in vitro test described above, see Folkman (1992) supra). For example, neovascularization of ischemic muscle can be demonstrated by experiments in which exogenously administered chimeric molecules of the invention augment collateral blood flow in experimentally induced mouse or rabbit hindlimb ischemia. See, e.g., Pu, L., et al., J. Invest. Surg. (1994) 7: 49-60; Couffinhal, T. et al., Am. J. Pathol. (1998) 152: 1667-1679; Witzenbichler, B., et al., Amer. J. Path. (1998)153: 381-394); and Bauters, C. et al., Circulation (1995) 91: 2802-2809); these references and all references cited therein are incorporated herein by reference. Endothelium-dependent relaxation of collateral microvessels after intramuscular gene transfer of VEGF have been shown in experimentally induced rat hindlimb ischemia. See, e.g., Takeshita, S. et al., Circulation (1998) 98: 1261-63; this reference and all references cited therein are incorporated herein by reference. Controlled, local delivery of VEGF from an osmotic pump was experimentally shown to promote neovascularization, limb perfusion, and functional improvements in a partially ischemic hindlimb rabbit model. See, e.g., Hopkins, S. et al., J. Vasc. Surg. (1998) 27: 886-894; this reference and all references cited therein are incorporated herein by reference. Experiments involving VEGF administration in a chronic porcine myocardial ischemia model have also been used. See, e.g. Harada, K. et al., Am. J. Physiol. (1996) 270: 886-94; this reference and all references cited therein are incorporated herein by reference.

The terms “ischemia,” “peripheral vascular disease,” “atherosclerosis,” and “coronary artery disease” as used herein, incorporates their common usages. These diseases, disorders or ailments can be modulated by VEGF or FGF, alone or in combination, in addition to other angiogenic factors. Ischemia is a condition characterized, for example, by a lack of oxygen supply in tissues of organs and limbs due to inadequate perfusion. Such inadequate perfusion can have number of natural causes, including atherosclerotic or restenotic lesions, anemia, or stroke, to name a few. Many medical interventions, such as the interruption of the flow of blood during bypass surgery, for example, also lead to ischemia. In addition to sometimes being caused by diseased cardiovascular tissue, ischemia can sometimes affect cardiovascular tissue, such as in ischemic heart disease. Ischemia can occur in any organ or limb, however, that is suffering a lack of oxygen supply.

The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, can cause myocardial ischemia and infarction in infancy, but this cause can be very rare in adults. Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, can be a rare cause of myocardial ischemia. Two or more causes of ischemia can coexist, such as an increase in oxygen demand due to left ventricular hypertrophy and a reduction in oxygen supply secondary to coronary atherosclerosis.

Cardiovascular disease refers to diseases of blood vessels of the heart. See. e.g., Kaplan, R. M., et al., “Cardiovascular diseases” in HEALTH AND HUMAN BEHAVIOR, pp. 206-242, (McGraw-Hill, New York 1993); this reference and all references cited therein are incorporated herein by reference. Cardiovascular disease can be generally one of several forms, including, e.g., hypertension (also referred to as high blood pressure), coronary heart disease, stroke, and rheumatic heart disease. Peripheral vascular disease refers to diseases of any of the blood vessels outside of the heart. It can be often a narrowing of the blood vessels that carry blood to leg and arm muscles.

The term “atherosclerosis” encompasses vascular diseases and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease.” The term “restenosis” refers to the renarrowing of the vascular lumen following vascular intervention, such as angioplasty and stent insertion. It can be clinically defined as a loss of initial luminal diameter gain. In hopes of reestablishing preangioplasty blood vessel diameter, the body attempts to remodel the vessel wall, stimulate new tissue growth which occupies space and re-occludes the lumen or stimulate tissue contraction. For example, during healing of the blood vessel after surgery, smooth muscle cells proliferate faster than endothelial cells narrowing the lumen of the blood vessel, and starting the atherosclerotic process anew.

The term “modulate” refers to the suppression, enhancement or induction of a function or condition. For example, the chimeric compounds of the invention can modulate angiogenesis by increasing blood vessel formation in ischemic heart tissue, thereby alleviating ischemia.

The term “treating” means the management and care of a human subject for the purpose of combating the disease, condition, or disorder and includes the administration of the chimeric molecule of the present invention to prevent the onset of the symptoms or complications, alleviating the symptoms or complications, or eliminating the disease, condition, or disorder.

The term “induce” or “induction” as used herein, refers to the activation, stimulation, enhancement, initiation and or maintenance of the cellular mechanisms or processes necessary for the formation of any of the tissue, repair process or development as described herein.

The term “library” means a collection of molecules. A library can contain a few or a large number of different molecules, varying from about ten molecules to several billion molecules or more. If desired, a molecule can be linked to a tag, which can facilitate recovery or identification of the molecule.

The term “molecule” is used broadly to mean an organic chemical such as a drug; a peptide, including a variant or modified peptide or peptide-like molecules such as a peptidomimetic or peptoid; or a protein such as an antibody or a growth factor receptor or a fragment thereof such as an Fv, Fc or Fab fragment of an antibody, which contains a binding domain. A molecule can be a nonnaturally occurring molecule, which does not occur in nature, but is produced as a result of in vitro methods, or can be a naturally occurring molecule such as a protein or fragment thereof expressed from a cDNA library.

A “chimeric molecule”, “chimeric protein”, “angiogenic chimeric molecule”, or “angiogenic chimeric protein” is a molecule that can have at least one binding site which recognizes the naturally-occurring cell surface angiogenic receptors, other tyrosine kinase receptors, or other receptors on the target cell or tissue and at least a second binding site which specifically binds to either normal or abnormal target cells or tissue.

A “fusion protein” refers to a composition comprising at least one polypeptide or peptide domain which is associated with a second domain. The second domain can be polypeptide, peptide, polysaccharide, or the like. The “fusion” can be an association generated by a peptide bond, a chemical linking, a charge interaction (e.g., electrostatic attractions, such as salt bridges, H-bonding), non covalent interaction, or the like. If the polypeptides are recombinant, the “fusion protein” can be translated from a common message. Alternatively, the compositions of the domains can be linked by any chemical or electrostatic means. The fusion proteins of the invention can also include linkers, epitope tags, enzyme cleavage recognition sequences, signal sequences, secretion signals, and the like.

The term “isolated,” when referring to a molecule or composition, such as the chimeric molecule or targeting molecule(s) of the invention, means that the chimeric molecule or targeting peptides are separated from at least one other compound, such as a protein, other nucleic acids (e.g., RNAs), or other contaminants with which it is associated in vivo or in its naturally occurring state. An isolated composition can, however, also be substantially pure. An isolated composition can be in a homogeneous state and can be in a dry or an aqueous solution. Purity and homogeneity can be determined, for example, using high performance liquid chromatography (HPLC). Thus, the isolated targeting molecule does not contain material normally associated with its in situ environment. Even where a protein has been isolated to a homogenous or dominant band, there are trace contaminants which co-purify with the desired protein.

“Administering” an expression vector, nucleic acid, an angiogenic factor, or a delivery vehicle to a cell comprises transducing, transfecting, electroporating, translocating, fusing, phagocytosing, shooting or ballistic methods, i.e., any means by which a protein or nucleic acid can be transported across a cell membrane and preferably into the nucleus of a cell.

A “delivery vehicle” refers to a compound, e.g., a liposome, toxin, or a membrane translocation polypeptide, which is used to administer a chimeric molecule of the invention. Delivery vehicles can also be used to administer nucleic acids encoding angiogenic factors, e.g., a lipid: nucleic acid complex, an expression vector, a virus, and the like.

The term “heterologous” is a relative term, which when used with reference to portions of a nucleic acid indicates that the nucleic acid comprises two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include an non-native (non-naturally occurring) nucleic acid that has integrated into the chromosome, or a nonnative (non-naturally occurring) extrachromosomal nucleic acid. In contrast, a naturally translocated piece of chromosome would not be considered heterologous in the context of this patent application, as it comprises an endogenous nucleic acid sequence that is native to the mutated cell.

Similarly, a heterologous protein indicates that the protein comprises two or more subsequences that are not found in the same relationship to each other in nature (e.g., a “fusion protein,” where the two subsequences are encoded by a single nucleic acid sequence). See, e.g., Current Protocols in Molecular Biology (Ausubel et al., (eds.) 1997; this reference and all references cited therein are incorporated herein by reference) for an introduction to recombinant techniques.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

The term “promoter” is defined as an array of nucleic acid control sequences that direct transcription. As used herein, a promoter typically includes necessary nucleic acid sequences near the start site of transcription, such as, in the case of certain RNA polymerase II type promoters, a TATA element, enhancer, CCAAT box, SP-1 site, etc. As used herein, a promoter also optionally includes distal enhancer or repressor elements, which can be located as much as several thousand base pairs from the start site of transcription. The promoters often have an element that is responsive to transactivation by a DNA-binding moiety such as a polypeptide, e.g., a nuclear receptor, Gal4, the lac repressor and the like.

The term “constitutive” promoter is a promoter that is active under most environmental and developmental conditions. An “inducible” promoter is a promoter that is active under certain environmental or developmental conditions.

The term “weak promoter” refers to a promoter having about the same activity as a wild type herpes simplex virus (“HSV”) thymidine kinase (“tk”) promoter or a mutated HSV tk promoter, as described in Eisenberg & McKnight, Mol. Cell. Biol. (1985) 5: 1940-1947.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell, and optionally integration or replication of the expression vector in a host cell. The expression vector can be part of a plasmid, virus, or nucleic acid fragment, of viral or non-viral origin. Typically, the expression vector includes an “expression cassette,” which comprises a nucleic acid to be transcribed operably linked to a promoter. The term expression vector also encompasses naked DNA operably linked to a promoter.

By “host cell” is meant a cell that contains a chimeric molecule of the invention or an expression vector or nucleic acid encoding a chimeric molecule of the invention. The host cell typically supports the replication or expression of the expression vector. Host cells can be prokaryotic cells such as E. coli, or eukaryotic cells such as yeast, fungal, protozoal, higher plant, insect, or amphibian cells, or mammalian cells such as CHO, HeLa, 293, COS-1, and the like, e.g., cultured cells (in vitro), explants and primary cultures (in vitro and ex vivo), and cells in vivo.

The term “nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiralmethyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide. The nucleotide sequences are displayed herein in the conventional 5′-3′ orientation.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Batzer et al., Nucleic Acid Res. (1991) 19: 5081; Ohtsuka et al., J. Biol. Chem. (1985) 260: 2605-2608; Rossolini et al., Mol. Cell. Probes (1994) 8: 91-98). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions or additions that alter, add or delete a single amino acid or nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art. Such conservatively modified variants are in addition to and do not exclude polymorphic variants and alleles of the invention.

The following groups each contain amino acids that are conservative substitutions for one another: 1) Alanine (A), Glycine (G); 2) Serine (S), Threonine (T); 3) Aspartic acid (D), Glutamic acid (E); 4) Asparagine (N), Glutamine (Q); 5) Cysteine (C), Methionine (M); 6) Arginine (R), Lysine (K), Histidine (H); 7) Isoleucine (I), Leucine (L), Valine (V); and 8) Phenylalanine (F), Tyrosine (Y), Tryptophan (W) (see, e.g., Creighton, Proteins (1984) for a discussion of amino acid properties).

The term “polynucleotide” is a nucleic acid of more than one nucleotide. A polynucleotide can be made up of multiple polynucleotide units that are referred to by description of the unit. For example, a polynucleotide can comprise a polynucleotide(s) having a coding sequence(s), a polynucleotide(s) that is a regulatory region(s) and/or other polynucleotide units commonly used in the art.

The term “biologically active fragment”, “biologically active form”, “biologically active equivalent” of and “functional derivative” of a wild-type angiogenic protein possesses a biological activity that is at least substantially equal to the biological activity of the wild type angiogenic protein. The above-mentioned terms are intended to include “fragments”, “mutants”, or “variants”, of the wild type angiogenic proteins. The term “fragment” is meant to refer to any polypeptide subset of the wild type angiogenic proteins. The term “mutant” is meant to refer to a molecule that can be substantially similar to the wild type form but possesses distinguishing biological characteristics. Such altered characteristics include but are not limited to altered substrate binding, altered substrate affinity and altered sensitivity to chemical compounds affecting biological activity of the angiogenic proteins or human angiogenic functional derivatives which can make the respective mutant attractive for targeted angiogenesis as disclosed herein. The term “variant” as described above, is refers to a molecule substantially similar in structure and function to either the entire wild-type protein or to a fragment thereof.

The term “gene” refers to a unit of inheritable genetic material found in a chromosome, such as in a human chromosome. Each gene is composed of a linear chain of deoxyribonucleotides which can be referred to by the sequence of nucleotides forming the chain. Thus, “sequence” is used to indicate both the ordered listing of the nucleotides which form the chain, and the chain which has that sequence of nucleotides. The term “sequence” is used in the same way in referring to RNA chains, linear chains made of ribonucleotides. The gene includes regulatory and control sequences, sequences which can be transcribed into an RNA molecule, and can contain sequences with unknown function. Some of the RNA products (products of transcription from DNA) are messenger RNAs (mRNAs) which initially include ribonucleotide sequences (or sequence) which are translated into a polypeptide and ribonucleotide sequences which are not translated. The sequences which are not translated include control sequences, introns and sequences with unknowns function. It can be recognized that small differences in nucleotide sequence for the same gene can exist between different persons, or between normal cells and cancerous cells, or between normal cells and diseased cells, without altering the identity of the gene.

The term “specific binding” (and equivalent phrases) refers to ability of a binding moiety (e.g., a receptor, antibody, or antiligand) to bind preferentially to a particular target molecule (e.g., ligand or antigen) in the presence of a heterogeneous population of proteins and other biologics (i.e., without significant binding to a other components present in a test sample). Typically, specific binding between two entities, such as a ligand and receptor, means a binding affinity of at least about 10⁶ M⁻¹, and preferably at least about 10⁷, 10⁸, 10⁹, or 10¹⁰ M⁻¹. In some embodiments specific binding is assayed (and specific binding molecules identified) according to the method of U.S. Pat. No. 5,622,699; this reference and all references cited therein are incorporated herein by reference). Typically a specific or selective reaction according to this assay is at least about twice background signal or noise and more typically at least about 5 or at least about 100 times background, or more.

When the binding moiety is an antibody, a variety of immunoassay formats can be used to select antibodies that are specifically immunoreactive with a particular protein. For example, solid-phase ELISA immunoassays are routinely used to select monoclonal antibodies specifically immunoreactive with an antigen. See Harlow and Lane (1988) Antibodies, A Laboratory Manual, Cold Spring Harbor Publications, New York, for a description of immunoassay formats and conditions that can be used to determine specific immunoreactivity (this reference and references cited therein are incorporated herein by reference).

“Specific hybridization” refers to the binding, duplexing, or hybridizing of a molecule only to a particular nucleotide sequence under stringent conditions when that sequence is present in a complex mixture (e.g., total cellular) DNA or RNA. Stringent conditions are conditions under which a probe can hybridize to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. (As the target sequences are generally present in excess, at Tₘ, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide or tetraalkyl ammonium salts. For example, conditions of 5× SSPE (750 mM NaCl, 50 mM Na Phosphate, 5 mM EDTA, pH 7.4) and a temperature of 25-30° C. are suitable for allele-specific probe hybridizations. (See Sambrook et al., Molecular Cloning 1989; this reference and all references cited therein are incorporated herein by reference).

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the composition.

The term “polysaccharide” or “oligosaccharide” incorporates its common usages, and includes, e.g., dextrose, glucose, lactose, mannose, mannan, and the like, as described below.

(1.) Targeted Angiogenesis

A. Targeting Vascular Endothelium

The present invention provides chimeric molecules comprising an angiogenic factor linked to a targeting molecule that specifically binds to a vascular endothelium.

Alterations in surface expression in the vasculature have been extensively studied in ischemia-reperfusion injury. See, e.g., Verrier, E., J. Cardiovasc Pharmacol. (1996) 27 Suppl 1: S26-30; Lefer, A. and Lefer, D., Cardiovasc Res (1996) 32: 743-51; Haller, H., Drugs (1997) 53 Suppl 1: 1-10; Kinlay, S. and Ganz, P. Am J Cardiol (1997) 80(9A): 111-161, and Luscher, T. et al., Ann. Rev Med (1993) 44: 395-418; these references and all references cited therein are incorporated herein by reference. In several organ systems, including the heart, kidney, brain and skeletal muscle, restoration of flow to a previously ischemic region induces an variety of responses. A variety of endothelial cell markers are known, including endothelial-leukocyte adhesion molecule (ELAM-1; Bevilacqua, M. et al., Proc. Natl. Acad. Sci. U.S.A. (1987) 84: 9238-9242); vascular cell adhesion molecule-1 (VCAM-1; Dustin, M. et al., J. Immunol. (1986) 137: 245-254); and intercellular adhesion molecule-1 (ICAM-1; Osborn, L. et al., Cell (1989) 59:1203-1211); These references and all references cited therein are incorporated herein by reference. The expression of these several cell adhesion molecules increases in a time-dependent manner and enhance leukocyte adhesion. The end result can be that reperfusion causes an inflammatory response and neutrophil recruitment that can accelerate cell death that can be associated with the ischemic injury. The immunoglobulin (“Ig”) supergene family receptors (ICAM-1, ICAM-2 and VCAM-1) are composed of variable numbers of repeated immunoglobulin-like domains (see, e.g., Williams, A. et al. Annu Rev Immunol (1988) 6: 381-387).

These alterations of surface expression in coronary vascular endothelium following ischemia-reperfusion injury can be used to isolate molecules that specifically bind to, for example, the cardiac vascular endothelium, using a variety of selection techniques.

B. Therapeutic Agents for Targeting Atherosclerosis and Restenosis

The vascular response to injury can involve an alteration in at least three fundamental cellular processes: cell growth, cell migration and extracellular matrix production. This vascular response to injury can be characteristic of the pathogenesis of various vascular diseases including, but not limited to, atherosclerosis, restenosis after angioplasty, vein bypass graft stenosis, prosthetic graft stenosis, angiogenesis and hypertension. For example, atherosclerotic lesions evolve as a result of vascular smooth muscle migration into the subintimal space, proliferation and the production of abundant extracellular matrix. Similarly, restenosis after angioplasty, vein bypass graft stenosis, prosthetic graft stenosis, angiogenesis and hypertension involve abnormalities in vascular cell growth, migration and matrix composition. See generally, Schwartz, D. et al., Thromb Haemost. (1995) 74: 541-51.

Atherosclerosis has been characterized by focal thickening of the inner portion of the artery wall, predisposing an individual to myocardial infarction (heart attack), cerebral infarction (stroke), hypertension (high blood pressure) and gangrene of the extremities. A common underlying event responsible for the formation of atherosclerotic lesions are the intimal thickening of proliferating smooth muscle cells in response to endothelial cell injury. “Intimal (or neointimal) hyperplasia or formation” means proliferation of arterial smooth muscle cells in the intima, in response to arterial endothelial denudation. Accumulation of smooth muscle cells in coronary arteries physically treated by angioplasty or by bypass surgery is also a prominent feature of restenosis. In addition to consisting primarily of proliferated smooth muscle cells, lesions of atherosclerosis are surrounded by large amounts of lipid-laden macrophages, varying numbers of lymphocytes and large amounts of connective tissue. PDGF is considered to be a principal growth-regulatory molecule responsible for smooth muscle cell proliferation (see, e.g., Dirks, R. et al., Mol Biol Rep (1995) 22: 1-24; this reference and all references cited therein are incorporated herein by reference). PDGF, therefore, can play a critical role in the atherosclerosis disease process (see, e.g. Hughes, A., Gen Pharmacol. (1996) 27:1079-89; this reference and all references cited therein are incorporated herein by reference). A number of other factors contribute to the pathophysiology of atherosclerosis and restenosis. These factors include, but are not limited to, angiotensin II, FGF, and transforming growth factor β1 (see, e.g., Pratt, R., J Am Soc Nephrol (1999) Suppl 11: S120-8; Gibbons, G., Am J Hypertens (1998) 11: 177S-181S; Cines, D. et al., Blood (1998) 91: 3527-61; O'Reilly et al., REGULATION OF ANGIOGENESIS, Goldberg & Rosen, Eds., (Birkhouser Verlag, Basel 1997), pp. 273-294; Saltis, J. et al., Clin Exp Pharmacol Physiol (1996) 23: 193-200; these references and all references cited therein are incorporated herein by reference).

Therapeutic agents that inhibit smooth muscle cell proliferation, endothelial cell proliferation and angiogenesis can be used with the chimeric molecules, methods, and gene therapy reagents of the invention. For example, one therapeutic agent, referred to as angiostatin (a naturally-occurring internal cleavage product of plasminogen) prohibits endothelial cell proliferation and is described in U.S. Pat. No. 5,733,876 (this reference is incorporated herein by reference). Another endothelial cell proliferation inhibitor includes endostatin, which is described in U.S. Pat. No. 5,854,205 (this reference is incorporated herein by reference). See, e.g., O'Reilly, M. et al., Cell (1997) 88: 277-85. Therapeutic agents such as angiostatin and endostatin, directed at the control of the angiogenic processes of atherosclerosis and restenosis as well as other angiogenesis-dependent (or angiogenic-related) diseases, can lead to the abrogation or mitigation of these diseases. See, e.g., Cao, Y. Prog Mol Subcell Biol. (1998) 20: 161-76; this reference and all references cited therein are incorporated herein by reference. Therefore, therapeutic agents that control the angiogenic processes of atherosclerosis and retenosis can be used in the chimeric molecules of the invention.

(2.) Selection and Preparation of Targeting Component for the Chimeric Molecules

(A) Identification of Targeting Molecules

Various methods are available for identifying and isolating molecules that specifically bind to certain cells and tissues such as vascular endothelium (e.g., the cardiac vascular endothelium). Some exemplary methods are described below.

(1) Phage Display Utilizing In Vivo Panning

In this method, molecules can be identified that specifically bind to one or a few selected organs by screening a library utilizing in vivo panning. The method is described in detail in U.S. Pat. No. 5,622,699 and is incorporated herein by reference. See also Pasqualini, R. et al., Nature (1996) 380: 364-366. An exemplary library for administering to a subject is a phage display peptide library. Phage display describes an in vitro or an in vivo selection technique in which a peptide or protein is genetically fused to a coat protein of a replicable genetic package, described below, resulting in display of the fused peptide or protein generally on the exterior of the replicable genetic package, while the DNA encoding the fusion generally resides within the replicable genetic package. This physical linkage between the displayed protein and the DNA encoding it allows screening of vast numbers of variants of the peptide or protein each linked to its corresponding DNA sequence.

Methods for preparing libraries containing diverse populations of various types of molecules such as peptides, polypeptides, proteins, fragments of protein, peptoids or peptidomimetics are well known in the art and commercially available. See, e.g., Lowman, H. et al., Ann. Rev Biophys Biomol Struct. (1997) 26: 401-24; Cortese, R. et al., Curr. Opin. Biotechnol. (1996) 7(6): 616-21; McGregor, D. et al., Mol Biotechnol. (1996) 6(2): 155-62; Ecker and Crooke, Biotechnology (1995) 13: 351-360 and Blondelle et al., Trends Anal. Chem. (1995) 14: 83-92, these references and all references cited therein, each of which is incorporated by reference. See also Goodman and Ro, Peptidomimetics for Drug Design, in BURGER'S MEDICINAL CHEMISTRY AND DRUG DISCOVERY, VOL. 1 (Wolff, M. E. (ed.). John Wiley & Sons 1995) and Gordon et al., J. Med. Chem. (1994) 37: 1385-1401, each of which is incorporated by reference.

Phage display technology can provide a means for expressing a diverse population of random or selectively randomized peptides. Various methods of phage display and methods for producing diverse populations of peptides are known (see, e.g., Ladner et al. U.S. Pat. No. 5,223,409; this reference and all references cited therein are incorporated herein by reference).

A replicable genetic package means a cell, spore or virus. The replicable genetic package can be eukaryotic or prokaryotic. A polypeptide display library is formed by introducing nucleic acids encoding exogenous polypeptides to be displayed into the genome of the replicable genetic package to form a fusion protein with an endogenous protein that is normally expressed from the outer surface of the replicable genetic package. Expression of the fusion protein, transport to the outer surface and assembly results in display of exogenous polypeptides from the outer surface of the genetic package.

The genetic packages most frequently used for display libraries are bacteriophage, particularly filamentous phage, and especially phage M13, Fd and F1. Most work has inserted libraries encoding polypeptides to be displayed into either gIII or gVIII of these phage forming a fusion protein (see, e.g., WO 91/19818; WO 91/18989; WO 92/01047 (gene III); WO 92/06204; and WO 92/18619 (gene VIII). Such a fusion protein comprises a signal sequence, usually from a secreted protein other than the phage coat protein, a polypeptide to be displayed and either the gene III or gene VIII protein or a fragment thereof. Exogenous coding sequences are often inserted at or near the N-terminus of gene III or gene VIII although other insertion sites are possible. Some filamentous phage vectors have been engineered to produce a second copy of either gene III or gene VIII. In such vectors, exogenous sequences are inserted into only one of the two copies. Expression of the other copy effectively dilutes the proportion of fusion protein incorporated into phage particles and can be advantageous in reducing selection against polypeptides deleterious to phage growth.

In another variation, exogenous polypeptide sequences are cloned into phagemid vectors which encode a phage coat protein and phage packaging sequences but which are not capable of replication. Phagemids are transfected into cells and packaged by infection with helper phage. Use of phagemid system also has the effect of diluting fusion proteins formed from coat protein and displayed polypeptide with wild-type copies of coat protein expressed from the helper phage (see, e.g., WO 92/09690).

Eukaryotic viruses can be used to display polypeptides in an analogous manner. For example, display of human heregulin fused to gp70 of Moloney murine leukemia virus has been reported by Han et al., Proc. Natl. Acad. Sci. USA (1995) 92: 9747-9751. Spores can also be used as replicable genetic packages. In this case, polypeptides are displayed from the outer surface of the spore. For example, spores from B. subtilis have been reported to be suitable. Sequences of coat proteins of these spores are provided by Donovan et al., J. Mol. Biol. (1987) 196: 1-10. Cells can also be used as replicable genetic packages. Polypeptides to be displayed are inserted into a gene encoding a cell protein that is expressed on the cells surface. Bacterial cells including Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli are preferred. Details of outer surface proteins are discussed by U.S. Pat. No. 5,571,698, and Georgiou et al., Nature Biotechnology (1997) 15: 29-34 and references cited therein.

Nucleic acids encoding polypeptides to be displayed by the polypeptide display library are inserted into the genome of a replicable genetic package by standard recombinant DNA techniques (see, e.g., Sambrook et al., Molecular Cloning, A Laboratory Manual (2d ed. 1989), incorporated herein by reference). The nucleic acids are ultimately expressed as polypeptides (with or without spacer or framework residues) fused to all or part of the an outer surface protein of the replicable package. Libraries often have sizes of about 10³, 10⁴, 10⁶, 10⁷, 10⁸ or more members.

These and other well known methods can be used to produce a phage display library, which can be subjected to the in vivo panning method described in U.S. Pat. No. 5,622,699 in order to identify peptides that selectively binds to one or a few selected organs and tissues. See, e.g., Pasqualini, R. and Ruoslahti, E. Nature (1996) 380: 362-366; Arap, W., et al., Science (1998) 279: 377-380; Rajotte, D. et al., J Clin Invest (1998) 102: 430-7; and Rajotte, D. et al., J Biol Chem. (1999) 274: 11593-8; these references are incorporated herein by reference. For example, in vivo selection or panning can be used to identify and isolate peptides that selectively bind normal cardiac endothelium or cardiac endothelium that has been altered by myocardial ischemia reperfusion injury. Similarly, normal or altered brain tissue can also be used to identify and isolate peptides that selectively bind to these tissues.

In general, a library of molecules, which contains a diverse population of random or selectively randomized molecules of interest, can be prepared, then 2.5×10⁸ transducing units (TU) of the phage libraries administered to a subject (e.g., intravenously through the jugular vein). After a preselected time allowing for phage circulation in vivo, the heart can be arrested by intraventricular injection of a hyperkalimic (30 mM KCl), hypothermic solution of DMEM, and the vasculature cleared of blood by perfusion with 5-10 mL of hyperkalemic DMEM through a left ventricular cannula. The heart and brain can then be harvested, homogenized, weighed and the phage rescued by standard techniques. For second and third rounds of selection, clones can be harvested from the previous round and individually grown to saturation. The cultures can then be pooled, the phage particles purified, then 10¹⁰ TU of this pool reinjected into similarly treated subjects. Phage ssDNA of individual clones from the third or more rounds can then be prepared and the inserts sequenced by standard techniques (see, e.g., Rojotte et al., supra). Phage with sequences appearing multiple times can then be characterized further by additional injections into similarly treated subjects. Subsequent rounds of screening can be performed to enrich for molecules that selectively bind to the organ of interest.

In vivo panning can also be used to identify phage that selectively target to altered vascular endothelium (i.e., cardiac endothelium). Vascular endothelium can be altered by myocardial ischemia-reperfusion injury. For example, thirty minutes of induced ischemia by standard procedures followed by thirty minutes of reperfusion (to allow for some changes to occur in the endothelium) can be used to alter the vascular endothelium. Cardiac tissue from animals that undergo the reperfusion injury can then be injected with phage. The in vivo panning procedure can then be performed as described above.

(2) Peptides on Plasmids

Another method is referred to as peptides on plasmids (“POPS”). See Schatz, P. et al. U.S. Pat. No. 5,733,731; these references and all references cited therein are incorporated herein by reference. Like the phage display methods, POPS employs a collection of pooled oligonucleotides encoding a diverse population of peptides, electroporation to generate a large library, and genetic linkage of peptides and oligonucleotides encoding them. However, POPS differs from the phage display method in that genetic linkage is not provided by a phage particle, but by expressing peptides with a DNA binding domain as a fusion protein that binds to a site on a vector encoding the fusion protein.

(3) Encoded Synthetic Library Method

A further method is referred to as the encoded synthetic library method (“ESL”). See U.S. Pat. No. 5,639,603; WO 95/12608, WO 93/06121, WO 94/08051, WO 95/35503 and WO 95/30642 (each of which is incorporated by reference for all purposes) In this method, the different compounds in the library are synthesized attached to separate supports (e.g., beads) by stepwise addition of the various components of the compounds in several rounds of coupling. A round of coupling can be performed by apportioning the supports between different reaction vessels and adding a different component to the supports in the different reaction vessels. The particular component added in a reaction vessel are recorded by the addition of a tag component to the support at a second site. Tag components can be oligonucleotides or other labels. If oligonucleotides are used, the correspondence tags and compounds are typically related by a correspondence regime other than the genetic code. After each round of synthesis, supports from the same reaction vessel can be apportioned between different reaction vessels and/or pooled with supports from another reaction vessel in the next round of synthesis. In any, and usually in all rounds of synthesis, the component added to the support can be recorded by addition of a further tag component at a second site of the support. After several rounds of synthesis, a large library of different compounds is produced in which the identities of compounds are encoded in tags attached to the respective supports bearing the compounds. The library can be screened for binding to a target. The ESL method can be used to produce libraries of any compound including peptides that can be synthesized in a component-by-component fashion.

The selection techniques described above can be used, for example, to target cardiac vascular endothelium, ischemic cardiac vascular endothelium, peripheral vascular endothelium, and ischemic peripheral vascular endothelium. The peripheral vascular endothelium is found in organs outside the heart and the limbs.

(B) Preferred Targeting Molecules

Preferred targeting molecules of the invention comprise an amino acid sequence selected from the group comprising GGGVFWQ, HGRVRPH, VVLVTSS, CLHRGNSC, and CRSWNKADNRSC using the in vivo panning procedure described above and referenced below. The GGGVFWQ, HGRVRPH, VVLVTSS, and CLHRGNSC peptides selectively bind to normal cardiac endothelium. More specifically, the GGGVFWQ peptide showed a 5-fold enrichment to normal cardiac vasculature, while the HGRVRPH, VVLVTSS, CLHRGNSC peptides showed a 2-fold enrichment to normal cardiac vasculature. The CRSWNKADNRSC peptide showed 5-fold enrichment to ischemic myocardium. Details of how these peptides were identified and their properties are described in U.S. Ser. No. ______ [Campbell & Flores LLP Attorney Docket # P-LJ 3512] filed on even date herewith which is specifically incorporated herein by reference.

(C) Selection/Preparation of Angiogenic Factor Component

Angiogenic factors have been described, supra. Exemplary angiogenic factors include, but are not limited to, VEGF polypeptides. An exemplary VEGF polypeptide, VEGF-B, has been isolated, cloned and sequenced. See Eriksson et al. U.S. Pat. No. 5,849,693; this reference are references cited therein are incorporated herein by reference. Presently, two isoforms of VEGF-B, generated by alternative splicing of mRNA, have been differentiated (Grimmond et al. 1996; Olfsson et al. 1996b; Townson et al. 1996; these references and all references cited therein are incorporated herein by reference). The two secreted forms of VEGF-B have 167 (VEGF-BI₆₇) and 186 (VEGFB₁₈₆) amino acid residues, respectively.

The VEGF-B₁₆₇ and VEGF-B₁₈₆ isoforms are produced as disulphide-linked homodimers with apparent molecular weights of 21 and 32 kD, respectively (Olofsson et al. 1996).

Once a VEGF polypeptide has been selected, designed, or otherwise provided, the VEGF polypeptide or the DNA encoding it are synthesized. Exemplary methods for synthesizing and expressing DNA encoding VEGF proteins are described below and in the Examples. The VEGF chimeric polypeptide or a polynucleotide encoding it can then be used to induce vascular proliferation.

VEGF proteins and nucleic acids encoding such VEGF proteins can be made using routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., supra); these references and all references cited therein are incorporated herein by reference). In addition, essentially any nucleic acid can be custom ordered from any of a variety of commercial sources. Similarly, peptides and antibodies can be custom ordered from any of a variety of commercial sources.

The nucleic acid encoding the angiogenic protein of choice can be typically cloned into intermediate vectors for transformation into prokaryotic or eukaryotic cells for replication and/or expression, e.g., for determination of K_{d}. Intermediate vectors are typically prokaryote vectors, e.g., plasmids, or shuttle vectors, or insect vectors, for storage or manipulation of the nucleic acid encoding angiogenic protein or production of protein. The nucleic acid encoding an angiogenic protein can also be typically cloned into an expression vector, for administration to a plant cell, animal cell, preferably a mammalian cell or a human cell, fungal cell, bacterial cell, or protozoal cell.

To obtain expression of a cloned gene or nucleic acid, a chimeric angiogenic protein can be typically subcloned into an expression vector that contains a promoter to direct transcription. Suitable bacterial and eukaryotic promoters are well known in the art and described, e.g., in Sambrook et al, Molecular Cloning, A Laboratory Manual (2nd ed. 1989); Kriegler, Gene Transfer and Expression: A Laboratory Manual (1990); and Current Protocols in Molecular Biology (Ausubel et al., supra); these reference and all references cited therein are incorporated herein by reference. Bacterial expression systems for expressing the angiogenic protein are available in, e.g., E. coli, Bacillus sp., and Salmonella (Palva et al., Gene I (1983) 22: 229-235). Kits for such expression systems are commercially available. Eukaryotic expression systems for mammalian cells, yeast, and insect cells are well known in the art and are also commercially available.

The promoter used to direct expression of a chimeric angiogenic protein nucleic acid depends on the particular application. For example, a strong constitutive promoter can be typically used for expression and purification of the angiogenic protein. In contrast, when an angiogenic protein is administered in vivo for gene regulation, either a constitutive or an inducible promoter can be used, depending on the particular use of the angiogenic protein. The promoter typically can also include elements that are responsive to transactivation, e.g., hypoxia response elements, Gal4 response elements, lac repressor response element, and small molecule control systems such as tet-regulated systems and the RU-486 system (see, e.g., Gossen & Bujard, Proc. Natl. Acad. Sci. U.S.A. (1992) 89: 5547; Oligino et al., Gene Ther. (1998) 5: 491-496; Wang et al., Gene Ther. (1997) 4: 432-441; Neering et al., Blood (1996) 88: 1147-1155; and Rendahl et al., Nat. Biotechnol. (1998) 16: 757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the angiogenic protein, and signals required, e.g., for efficient polyadenylation of the transcript, transcriptional termination, ribosome binding sites, or translation termination. Additional elements of the cassette can include, e.g., enhancers, and heterologous spliced intronic signals.

The particular expression vector used to transport the genetic information into the cell can be selected with regard to the intended use of the angiogenic protein, e.g., expression in plants, animals, bacteria, fungus, and protozoa. Standard bacterial expression vectors include plasmids such as pBR322 based plasmids, pSKF, pET23D, and commercially available fusion expression systems such as GST and LacZ. These fusion proteins can be used for purification of the angiogenic protein. Epitope tags can also be added to recombinant proteins to provide convenient methods of isolation, for monitoring expression, and for monitoring cellular and subcellular localization.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with an angiogenic protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., J. Biol. Chem. (1989) 264: 17619-17622; Guide to Protein Purification, in Methods in Enzymology, Vol. 182 (Deutscher, ed., 1990). Transformation of eukaryotic and prokaryotic cells are performed according to standard techniques (see, e.g., Morrison, J. Bact. (1977) 132: 349-351; Clark-Curtiss & Curtiss, Methods in Enzymology 101: 347-362 (Wu et al., eds. 1983).

(D) Coupling of Targeting Component to Angiogenic Factor Component

Chimeric molecules of the present invention include at least two components: a functional angiogenic factor and a targeting molecule. The functional angiogenic factor can comprise, for example, an amino acid or polypeptide sequence which binds an angiogenic factor receptor on endothelial cells or contains a sequence which will affect the target tissue in a specific way. The targeting molecule can comprise an amino acid or polypeptide sequence which binds to one or more types of vascular endothelial cells. The amino acid sequence which is the functional angiogenic factor can be a ligand binding domain of the angiogenic factor receptor; the amino acid sequence which is the targeting molecule can bind to a cell-surface receptor and can be thus a cell surface receptor ligand.

In the case in which the selected substance is a normally-occurring constituent of the blood, lymph, or extracellular fluid, the ligand-binding domain which binds the angiogenic factor receptor is an amino acid sequence which normally binds the angiogenic factor receptor (i.e., binds the selected angiogenic factor receptor in humans). A modified form of such a sequence with altered binding properties, or an amino acid sequence which is not usually found in humans but has been produced by synthetic or genetic engineering methods and also can bind the selected angiogenic factor receptor. For example, the angiogenic factor and/or targeting molecule can be amino acid sequences selected from a combinatorial peptide library or phage display library. The angiogenic factor and/or targeting molecules can also comprise the antigen binding domain of an immunoglobulin or single-chain antibody, wherein the antigen binding domain of the immunoglobulin or single-chain antibody recognizes the desired selected substance or cell surface receptor. In the case in which the selected substance is a foreign constituent, the amino acid sequence which binds the selected substance can be one selected from naturally-occurring ligand-binding domains which bind the foreign constituent or an amino acid sequence designed to bind the foreign constituent.

The domains of the chimeric protein can be linked in a variety of configurations, as long as the resulting chimeric protein is able to bind both the vascular endothelial growth factor receptor and the targeting molecule receptor. One configuration could be in the form of a fusion protein. A “fusion protein” refers to a composition comprising at least one polypeptide or peptide domain which is associated with a second domain. The second domain can be polypeptide, peptide, polysaccharide, or the like. The “fusion” can be an association generated by a peptide bond, a chemical linking, a charge interaction (e.g. electrostatic attractions, such as salt bridges, H-bonding) noncovalent interactions or the like. If the polypeptides are recombinant, the “fusion protein” can be translated from a common message. Alternatively, the compositions of the domains can be linked by any chemical or electrostatic means. The fusion proteins of the invention can also include linkers, epitope tags, enzyme cleavage recognition sequences, signal sequences, secretion signals, and the like.

Typically, the two domains are encoded by a single reading frame in a recombinant DNA molecule, and the two domains are linked by a peptide bond. The two domains can be separated by one or more amino acids also encoded by the open reading frame. Alternatively, the two domains can be expressed from separate DNA molecules and become linked in vitro or in vivo through either non-covalent (e.g., hydrophobic or ionic interaction) or covalent (e.g., disulfide) linkage. In addition, methods have been described for producing biologically active peptide dimers. See, e.g., EP 0721983 A1, which is incorporated herein by reference.

(3.) Formulation and Administration of Chimeric Molecules: Pharmaceutical Compositions

(A) Protein-based Therapeutics

The angiogenic factor chimeric molecules of the invention can be typically combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptide complexes, or excipients or other stabilizers and/or buffers. Detergents can also used to stabilize or to increase or decrease the absorption of the pharmaceutical composition, see infra for exemplary detergents, including liposomal carriers. Pharmaceutically acceptable carriers and formulations for peptides and polypeptide are known to the skilled artisan and are described in detail in the scientific and patent literature, see, e.g., Remington's, supra, and Banga, A. K., Therapeutic Peptides and Proteins. Formulation, Processing and Delivery Systems (1996) (Technomic Publishing AG, Basel, Switzerland); these references and references cited therein are incorporated herein by reference.

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the protein or polypeptide of the invention and on its particular physiochemical characteristics.

(1) Aqueous Solutions for Enteral, Parenteral Or Transmucosal Administration

The compositions for administration will commonly comprise a solution of the peptide or polypeptide of the invention dissolved in a pharmaceutically acceptable carrier, preferably an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of the chimeric molecule in these formulations can vary widely, and will be selected primarily based on fluid volumes, viscosities, body weight and the like in accordance with the particular mode of administration selected and the patient's needs.

(2) Solid Formulations for Enteral Delivery

Solid formulations can be used for enteral (oral) administration. They can be formulated as, e.g., pills, tablets, powders or capsules. For solid compositions, conventional nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (chimeric molecule). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like.

It is recognized that the chimeric molecule of the invention, when administered orally, must be protected from digestion. This is typically accomplished either by complexing the peptide or polypeptide complex with a composition to render it resistant to acidic and enzymatic hydrolysis or by packaging the peptide or complex in an appropriately resistant carrier such as a liposome. Means of protecting compounds from digestion are well known in the art, see, e.g., Fix Pharm Res. (1996) 13: 1760-1764; Samanen J. Pharm. Pharmacol. (1996) 48: 119-135; U.S. Pat. No. 5,391,377, describing lipid compositions for oral delivery of therapeutic agents (liposomal delivery is discussed in further detail, infra).

(3) Topical Formulations For Transdermal/Transmucosal Delivery

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Banga, Chapt. 10; Sayani “Systemic delivery of peptides and proteins across absorptive mucosae” Crit. Rev. Ther. Drug Carrier Syst. (1996) 13: 85-184. For topical, transdermal administration, the agents are formulated into ointments, creams, salves, powders and gels. Transdermal delivery systems can also include, e.g., patches. See, e.g., Banga, Chapt. 9.

The peptides and polypeptide complexes can also be administered in sustained delivery or sustained release mechanisms, which can deliver the formulation internally. For example, biodegradeable microspheres or capsules or other biodegradeable polymer configurations capable of sustained delivery of a composition (e.g., a chimeric molecule) can be included in the formulations of the invention (see, e.g., Putney Nat. Biotechnol. (1998) 16: 153-157).

(4) Formulations for Inhalation Delivery

For inhalation, the peptide or polypeptide can be delivered using any system known in the art, including dry powder aerosols, liquids delivery systems, air jet nebulizers, propellant systems, and the like. See, e.g., Patton et al., Biotechniques (1998) 16: 141-143; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like.

For example, the pharmaceutical formulation can be administered in the form of an aerosol or mist. For aerosol administration, the formulation can be supplied in finely divided form along with a surfactant and propellant. The surfactant preferably is soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride such as, for example, ethylene glycol, glycerol, erythritol, arabitol, mannitol, sorbitol, the hexitol anhydrides derived from sorbitol, and the polyoxyethylene and polyoxypropylene derivatives of these esters. Mixed esters, such as mixed or natural glycerides can be employed. The surfactant can constitute 0.1% to 20% by weight of the composition, preferably 0.25% to 5%. The balance of the formulation is ordinarily propellant. Liquefied propellants are typically gases at ambient conditions, and are condensed under pressure. Among suitable liquefied propellants are the lower alkanes containing up to 5 carbons, such as butane and propane; and preferably fluorinated or fluorochlorinated alkanes. Mixtures of the above can also be employed. In producing the aerosol, a container equipped with a suitable valve is filled with the appropriate propellant, containing the finely divided compounds and surfactant. The ingredients are thus maintained at an elevated pressure until released by action of the valve. See, e.g., Edwards et al., Science (1997) 276: 1868-1871.

In another embodiment, the device for delivering the formulation to respiratory tissue is an inhaler in which the formulation vaporizes. Other liquid delivery systems include, e.g., air jet nebulizers.

(5) Other Formulations

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the complexes in vesicles composed of substances such as proteins, lipids (for example, liposomes, see below), carbohydrates, or synthetic polymers (discussed above). For a general discussion of pharmacokinetics, see, e.g., Remington's, Chapters 37-39, or Banga, Chapt. 6. See also Lee, P. I. D. et al., Pharmacokinetic Analysis. A Practical Approach (Technomic Publishing AG, Basel, Switzerland 1996)

(6) Routes of Delivery

The peptide and polypeptide complexes used in the methods of the invention can be delivered alone or as pharmaceutical compositions by any means known in the art, e.g., systemically, regionally, or locally; by intraarterial, intrathecal (IT), intravenous (IV), intramuscular injection, parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's or Banga. Particularly preferred modes of administration include intra-arterial, intramuscular injections or intrathecal (IT) injections, especially when it is desired to have a “regional effect,” e.g., to focus on a specific organ, e.g., brain and CNS (see e.g., Gurun Anesth Analg. (1997) 85: 317-323) and the heart. For example, intra-carotid artery injection if preferred where it is desired to deliver a peptide or polypeptide complex of the invention directly to the brain. Parenteral administration is a preferred route of delivery if a high systemic dosage is needed. Enteral administration is a preferred method if administration of peptide to induce oral tolerance is the therapeutic objective, see, e.g., Kennedy J. Immunol. (1997) 159: 1036-1044; Kent Ann. NY Acad. Sci. (1997) 815: 412-422. Actual methods for preparing parenterally administrable compositions will be known or apparent to those skilled in the art and are described in detail, in e.g., Remington's, Banga Chapt 7. See also, Bai J. Neuroimmunol. (1997) 80: 65-75; Warren J. Neurol. Sci. (1997) 152: 31-38; Tonegawa J. Exp. Med. (1997) 186: 507-515.

(7) Treatment Regimens: Pharmacokinetics

The pharmaceutical compositions can be administered in a variety of unit dosage forms depending upon the method of administration. Dosages for typical peptide and polypeptide pharmaceutical compositions are well known to those of skill in the art. Such dosages are typically advisorial in nature and are adjusted depending on the particular therapeutic context, patient tolerance, etc. The amount of the chimeric molecule adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., Remington's; Egleton Peptides (1997) 18: 1431-1439; Langer Science (1990) 249: 1527-1533.

In therapeutic applications, compositions are administered to a patient suffering from ischemic disease in an amount sufficient to cure or at least partially arrest the disease and/or its complications. An amount adequate to accomplish this is defined as a “therapeutically effective dose.” Amounts effective for this use will depend upon the severity of the disease, general state of the patient's health, frequency and routes of administration, clinician's judgment, and the like.

Dosages can be determined empirically, by assessing the abatement or amelioration of symptoms, or by objective criteria, such analysis of blood or histopathology specimens. Thus, the compositions of the invention are administered to arrest the progress of the disease and to reduce the onset, frequency or severity of these or other symptoms.

The pharmaceutical compositions containing the peptide and complexes of the invention can be administered alone or in conjunction with other therapeutic treatments. Single or multiple administrations of the compositions can be administered depending on the dosage and frequency as required and tolerated by the patient.

(8) Liposomal Formulations

The invention provides pharmaceuticals for formulations in which the chimeric molecules are incorporated in lipid monolayers or bilayers. The invention also provides formulations in which water soluble peptides or complexes have been attached to the surface of the monolayer or bilayer. For example, peptides can be attached to hydrazide-PEG-(distearoylphosphatidyl) ethanolamine-containing liposomes (see, e.g., Zalipsky Bioconjug. Chem. (1995) 6: 705-708). Liposomes or any form of lipid membrane, such as planar lipid membranes or the cell membrane of an intact cell, e.g. a red blood cell, can be used. Liposomal formulations can be by any means, including administration intravenously, transdermally (see, e.g., Vutla J. Pharm. Sci. (1996) 85: 58), transmucosally, or orally. The invention also provides pharmaceutical preparations in which the peptides and/or complexes of the invention are incorporated within micelles and/or liposomes (see, e.g., Suntres J. Pharm. Pharmacol. (1994) 46: 23-28; Woodle Pharm. Res. (1992) 9: 260-265).

Liposomes and liposomal formulations can be prepared according to standard methods and are also well known in the art, see, e.g., Remington's; Akimaru Cytokines Mol. Ther. (1995) 1: 197-210; Alving Immunol. Rev. (1995) 145: 5-31; Szoka Ann. Rev. Biophys. Bioeng. (1980) 9: 467, U.S. Pat. Nos. 4, 235,871, 4,501,728 and 4,837,028; these references and all references cited therein are incorporated herein by reference. In one embodiment, liposomes of the present invention typically contain the chimeric molecule complex positioned on the surface of the liposome in such a manner that the complexes are available for interaction with the receptors on endothelial cells. U.S. Pat. No. 5,876,747 describes liposomes that preferentially travel to cardiac and skeletal muscles and is incorporated herein by reference.

Liposome charge is an important determinant in liposome clearance from the blood, with negatively charged liposomes being taken up more rapidly by the reticuloendothelial system (Juliano, Biochem. Biophys. Res. Commun. (1975) 63: 651) and thus having shorter half-lives in the bloodstream. Incorporating phosphatidylethanolamine derivatives enhance the circulation time by preventing liposomal aggregation. For example, incorporation of N-(omega-carboxy)acylamidophosphatidylethanolamines into large unilamellar vesicles of L-alpha-distearoylphosphatidylcholine dramatically increases the in vivo liposomal circulation lifetime (see, e.g., Ahl Biochim. Biophys. Acta (1997) 1329: 370-382). Liposomes with prolonged circulation half-lives are typically desirable for therapeutic and diagnostic uses. For instance, liposomes which can be maintained from 8, 12, or up to 24 hours in the bloodstream are particularly preferred embodiments of the invention.

Typically, the liposomes are prepared with about 5 to 15 mole percent negatively charged phospholipids, such as phosphatidylglycerol, phosphatidylserine or phosphatidyl-inositol. Added negatively charged phospholipids, such as phosphatidylglycerol, also serve to prevent spontaneous liposome aggregating, and thus minimize the risk of undersized liposomal aggregate formation. Membrane-rigidifying agents, such as sphingomyelin or a saturated neutral phospholipid, at a concentration of at least about 50 mole percent, and 5 to 15 mole percent of monosialylganglioside, can provide increased circulation of the liposome preparation in the bloodstream, as generally described in U.S. Pat. No. 4,837,028.

Additionally, the liposome suspension can include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

The formulations of the invention can include multilamellar vesicles of heterogeneous sizes. In this method, the vesicle-forming lipids are dissolved in a suitable organic solvent or solvent system and dried under vacuum or an inert gas to form a thin lipid film. If desired, the film can be redissolved in a suitable solvent, such as tertiary butanol, and then lyophilized to form a more homogeneous lipid mixture which is in a more easily hydrated powderlike form. This film is covered with an aqueous solution of the peptide or polypeptide complex and allowed to hydrate, typically over a 15 to 60 minute period with agitation. The size distribution of the resulting multilamellar vesicles can be shifted toward smaller sizes by hydrating the lipids under more vigorous agitation conditions or by adding solubilizing detergents such as deoxycholate. The hydration medium contains the peptide or complex at a concentration which is desired in the interior volume of the liposomes in the final liposome suspension. Typically the drug solution contains between 10 to 100 mg/ml of the peptides or complexes of the invention in a buffered saline solution.

Following liposome preparation, the liposomes can be sized to achieve a desired size range and relatively narrow distribution of liposome sizes. One preferred size range is about 0.2 to 0.4 microns, which allows the liposome suspension to be sterilized by filtration through a conventional filter, typically a 0.22 micron filter. The filter sterilization method can be carried out on a high through-put basis if the liposomes have been sized down to about 0.2 to 0.4 microns. Several techniques are available for sizing liposome to a desired size (see, e.g., U.S. Pat. No. 4,737,323). Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size discrimination. Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes can be extruded through successively smaller-pore membranes, to achieve a gradual reduction in liposome size.

Even under the most efficient encapsulation methods, the initial sized liposome suspension can contain up to 50% or more complex in a free (nonencapsulated) form. Several methods are available for removing non-entrapped compound from a liposome suspension, if desired for a particular formulation. In one method, the liposomes in the suspension are pelted by high-speed centrifugation leaving free compound and very small liposomes in the supernatant. Another method involves concentrating the suspension by ultrafiltration, then resuspending the concentrated liposomes in a replacement medium. Alternatively, gel filtration can be used to separate large liposome particles from solute molecules. Following this treatment, the liposome suspension can be brought to a desired concentration for use in, e.g., an intravenous, IP, transdermal, or transmucosal administration. This involve resuspending the liposomes in a suitable volume of appropriate medium, where the liposomes have been concentrated, for example by centrifugation or ultrafiltration, or concentrating the suspension, where the drug removal step has increased total suspension volume. The suspension is then sterilized by filtration as described above. These liposomes comprising the peptides or chimeric molecule can be administered parenterally or locally in a dose which varies according to, e.g., the manner of administration, the drug being delivered, the particular disease being treated.

Micelles are commonly used in the art to increase solubility of molecules having nonpolar regions. One of skill will thus recognize that micelles are useful in compositions of the present invention. Micelles comprising the complexes of the invention are prepared according to methods well known in the art (see, e.g., Remington's, Chap. 20). Micelles comprising the peptides and/or complexes of the present invention are typically prepared using standard surfactants or detergents. Micelles are formed by surfactants (molecules that contain a hydrophobic portion and one or more ionic or otherwise strongly hydrophilic groups) in aqueous solution. As the concentration of a solid surfactant increases, its monolayers adsorbed at the air/water or glass/water interfaces become so tightly packed that further occupancy requires excessive compression of the surfactant molecules already in the two monolayers. Further increments in the amount of dissolved surfactant beyond that concentration cause amounts equivalent to the new molecules to aggregate into micelles. Suitable surfactants include sodium laureate, sodium oleate, sodium lauryl sulfate, octaoxyethylene glycol monododecyl ether, octoxynol 9 and PLURONIC F-127® (Wyandotte Chemicals Corp.). Preferred surfactants are nonionic polyoxyethylene and polyoxypropylene detergents compatible with IV injection such as PLURONIC F-127®, n-octyl-alpha-D-glucopyranoside, and the like. Phospholipids, such as those described for use in the production of liposomes, can also be used for micelle formation. Mixed micelles can be formed in the presence of common surfactants or phospholipids and the subunits. The mixed micelles of the present invention can comprise any combination of the subunits, phospholipids and/or surfactants. Thus, the micelles can comprise subunits and detergent, subunits in combination with both phospholipids and detergent, or subunits and phospholipid.

(B) Nucleic Acid Based Therapeutics

Broadly speaking, a gene therapy vector is an exogenous polynucleotide which produces a medically useful phenotypic effect upon the mammalian cell(s) into which it is transferred. A vector can or can not have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA. Vectors used in gene therapy can be viral or nonviral. Viral vectors are usually introduced into a patient as components of a virus. Nonviral vectors, typically dsDNA, can be transferred as naked DNA or associated with a transfer-enhancing vehicle, such as a receptor-recognition protein, lipoamine, or cationic lipid.

(1) Viral-Based Methods

Viral vectors, such as retroviruses, adenoviruses, adenoassociated viruses and herpes viruses, are often made up of two components, a modified viral genome and a coat structure surrounding it (see generally Smith et al., Ann. Rev. Microbiol. (1995) 49, 807-838; this reference and all references cited therein are incorporated herein by reference), although sometimes viral vectors are introduced in naked form or coated with proteins other than viral proteins. Most current vectors have coat structures similar to a wildtype virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

(a) Retroviruses

Retroviruses comprise a large class of enveloped viruses that contain single—stranded RNA as the viral genome. During the normal viral life cycle, viral RNA is reverse-transcribed to yield double-stranded DNA that integrates into the host genome and is expressed over extended periods. As a result, infected cells shed virus continuously without apparent harm to the host cell. The viral genome is small (approximately 10 kb), and its prototypical organization is extremely simple, comprising three genes encoding gag, the group specific antigens or core proteins; pol, the reverse transcriptase; and env, the viral envelope protein. The termini of the RNA genome are called long terminal repeats (LTRs) and include promoter and enhancer activities and sequences involved in integration. The genome also includes a sequence required for packaging viral RNA and splice acceptor and donor sites for generation of the separate envelope mRNA. Most retroviruses can integrate only into replicating cells, although human immunodeficiency virus (HIV) appears to be an exception. This property restricts the use of retroviruses as vectors for gene therapy.

Retrovirus vectors are relatively simple, containing the 5′ and 3′ LTRs, a packaging sequence, and a transcription unit composed of the gene or genes of interest, which is typically an expression cassette. To grow such a vector, one must provide the missing viral functions in trans using a so-called packaging cell line. Such a cell is engineered to contain integrated copies of gag, pol, and env but to lack a packaging signal so that no helper virus sequences become encapsidated. Additional features added to or removed from the vector and packaging cell line reflect attempts to render the vectors more efficacious or reduce the possibility of contamination by helper virus.

The main advantage of retroviral vectors is that they integrate and are therefore potentially capable of long-term expression. They can be grown in relatively large amounts, but care is needed to ensure the absence of helper virus.

(b) Adenoviruses

Adenoviruses comprise a large class of nonenveloped viruses containing linear double-stranded DNA. The normal life cycle of the virus does not require dividing cells and involves productive infection in permissive cells during which large amounts of virus accumulate. The productive infection cycle takes about 32-36 hours in cell culture and comprises two phases, the early phase, prior to viral DNA synthesis, and the late phase, during which structural proteins and viral DNA are synthesized and assembled into virions. In general, adenovirus infections are associated with mild disease in humans.

Adenovirus vectors are somewhat larger and more complex than retrovirus or AAV vectors, partly because only a small fraction of the viral genome is removed from most current vectors. If additional genes are removed, they are provided in trans to produce the vector, which so far has proved difficult. Instead, two general types of adenovirus-based vectors have been studied, E3-deletion and E1-deletion vectors. Some viruses in laboratory stocks of wildtype lack the E3 region and can grow in the absence of helper. This ability does not mean that the E3 gene products are not necessary in the wild, only that replication in cultured cells does not require them. Deletion of the E3 region allows insertion of exogenous DNA sequences to yield vectors capable of productive infection and the transient synthesis of relatively large amounts of encoded protein.

Deletion of the E1 region disables the adenovirus, but such vectors can still be grown because there exists an established human cell line (called “293”) that contains the E1 region of Ad5 and that constitutively expresses the E1 proteins. Most recent gene therapy applications involving adenovirus have utilized E1 replacement vectors grown in 293 cells.

The main advantages of adenovirus vectors are that they are capable of efficient episomal gene transfer in a wide range of cells and tissues and that they are easy to grow in large amounts. The main disadvantage is that the host response to the virus appears to limit the duration of expression and the ability to repeat dosing, at least with high doses of first-generation vectors.

(c) Adeno-Associated Virus (AAV)

AAV is a small, simple, nonautonomous virus containing linear single-stranded DNA. See Muzycka, Current Topics Microbiol. Immunol. (1992) 158, 97-129; this reference and all references cited therein are incorporated herein by reference. The virus requires co-infection with adenovirus or certain other viruses in order to replicate. AAV is widespread in the human population, as evidenced by antibodies to the virus, but it is not associated with any known disease. AAV genome organization is straightforward, comprising only two genes: rep and cap. The termini of the genome comprises terminal repeats (ITR) sequences of about 145 nucleotides.

AAV-based vectors typically contain only the ITR sequences flanking the transcription unit of interest. The length of the vector DNA cannot greatly exceed the viral genome length of 4680 nucleotides. Currently, growth of AAV vectors is cumbersome and involves introducing into the host cell not only the vector itself but also a plasmid encoding rep and cap to provide helper functions. The helper plasmid lacks ITRs and consequently cannot replicate and package. In addition, helper virus such as adenovirus is often required. The potential advantage of AAV vectors is that they appear capable of long-term expression in nondividing cells, possibly, though not necessarily, because the viral DNA integrates. The vectors are structurally simple, and they can therefore provoke less of a host-cell response than adenovirus. A major limitation at present is that AAV vectors are extremely difficult to grow in large amounts.

(2) Non-Viral Gene Transfer Methods

Nonviral nucleic acid vectors used in gene therapy include plasmids, RNAs, antisense oligonucleotides (e.g., methylphosphonate or phosphorothiolate), polyamide nucleic acids, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Gene therapy vectors of all kinds can also include a selectable marker gene. Examples of suitable markers include, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg, Proc. Natl. Acad. Sci. U.S.A. (1981) 78, 2072; Southern & Berg, J. Mol. Appl. Genet. (1982) 1, 327).

Before integration, the vector has to cross many barriers which can result in only a very minor fraction of the DNA ever being expressed. Limitations to high level gene expression include: loss of vector due to nucleases present in blood and tissues; inefficient entry of DNA into a cell; inefficient entry of DNA into the nucleus of the cell and preference of DNA for other compartments; lack of DNA stability in the nucleus (factor limiting nuclear stability can differ from those affecting other cellular and extracellular compartments), efficiency of integration into the chromosome; and site of integration.

These potential losses of efficiency can be addressed by including additional sequences in a nonviral vector besides the expression cassette from which the product effecting therapy is to be expressed. The additional sequences can have roles in conferring stability both outside and within a cell, mediating entry into a cell, mediating entry into the nucleus of a cell and mediating integration within nuclear DNA. For example, aptamer-like DNA structures, or other protein binding sites can be used to mediate binding of a vector to cell surface receptors or to serum proteins that bind to a receptor thereby increasing the efficiency of DNA transfer into the cell.

Other DNA sequences can directly or indirectly result in avoidance of certain compartments and preference for other compartments, from which escape or entry into the nucleus is more efficient. Other DNA sites and structures directly or indirectly bind to receptors in the nuclear membrane or to other proteins that go into the nucleus, thereby facilitating nuclear uptake of a vector. Other DNA sequences directly or indirectly affect the efficiency of integration. For integration by homologous recombination, important factors are the degree and length of homology to chromosomal sequences, as well as the frequency of such sequences in the genome (e.g., alu repeats). The specific sequence mediating homologous recombination is also important, since integration occurs much more easily in transcriptionally active DNA. Methods and materials for constructing homologous targeting constructs are described by e.g., Mansour et a., Nature (1988) 336: 348; Bradley et al., Bio/Technology (1992) 10: 534.

For nonhomologous, illegitimate and site-specific recombination, recombination is mediated by specific sites on the therapy vector which interact with cell encoded recombination proteins (e.g., cre/lox and flp/frt systems). For example Baubonis & Sauer, Nuc. Acids Res. (1993) 21, 2025-2029 report that a vector including a loxP site becomes integrated at a loxP site in chromosomal DNA in the presence of cre enzyme.

Nonviral vectors encoding products useful in gene therapy can be introduced into an animal by means such as lipofection, biolistics, virosomes, liposomes, immunoliposomes, polycation: nucleic acid conjugates, naked DNA, artificial virions, agent-enhanced uptake of DNA, ex vivo transduction. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024.

Unlike existing viral-based gene therapy vectors which can only incorporate a relatively small non-viral polynucleotide sequence into the viral genome because of size limitations for packaging virion particles, naked DNA or lipofection complexes can be used to transfer large (e.g., 50-5,000 kb) exogenous polynucleotides into cells. This property of nonviral vectors is particularly advantageous since many genes which can be delivered by therapy span over 100 kilobases (e.g., amyloid precursor protein (APP) gene, Huntington's chorea gene) and large homologous targeting constructs or transgenes can be required for efficient integration. Optionally, such large genes can be delivered to target cells as two or more fragments and reconstructed by homologous recombination within a cell (see WO 92/03917).

(C) Applications of Gene Therapy

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

(4.) Therapeutic Kits

Kits can be supplied for therapeutic or diagnostic uses. In one embodiment the pharmaceutical formulation of the invention is in a lyophilized form, which can be placed in a container. The complexes, which can also be conjugated to a label, or unconjugated, are included in the kits with buffers, such as Tris, phosphate, carbonate, stabilizers, biocides, inert proteins, e.g., serum albumin, or the like, and a set of instructions for use. Generally, these materials will be present in less than about 5% wt. based on the amount of complex and usually present in total amount of at least about 0.001% wt. based again on the protein concentration. Frequently, it will be desirable to include an inert extender or excipient to dilute the active ingredients, where the excipient can be present in from about 1% to 99% wt. of the total composition. Where an antibody capable of binding to the complex is employed in an assay, this will usually be present in a separate vial. The antibody is typically conjugated to a label and formulated according to techniques well known in the art.

### EXAMPLES

The following examples are offered to illustrate, but no to limit the claimed invention.

### Example 1

Expression of Wild-type VEGF-B₁₆₇ in CHO Cells

The VEGF-B₁₆₇ splice variant of VEGF-B, after expression and secretion from mammalian cells, is a non-glycosylated and cell-associated antiparallel dimer that displays mitogenic activity in endothelial cells (see, e.g., Eriksson, U., and K. Alitalo Curr Top Microbiol Immunol. (1999) 237: 41-57). The wild-type (wt) VEGF-B₁₆₇ molecule is expressed in Chinese hamster ovary (CHO) cells and further used for chemical coupling with the targeting peptides of the invention. CHO cells were chosen as the production host because correct folding and dimerization of cys-rich proteins occurs preferentially in mammalian cells. Expression in E. coli or the yeast pichia pastoris can be an alternative procedure and can require solubilization of inclusion bodies in denaturants and refolding.

Construction of the Plasmid pVEGF-Bwt167 and Expression of VEGF-B₁₆₇ in CHO Cells.

The plasmid is constructed as described in Materials and Methods below. In this plasmid, the VEGF-B₁₆₇ cDNA is controlled by the SV40 early promoter. Immediately upstream of the ATG initiation codon, the DNA sequence is changed into the optimal context for initiation of translation in eukaryotic cells. Cotransfection of the plasmids pVEGF-Bwt167 and pSV-rdhfr (that contains the murine DHFR selection marker) into dhfr-deficient CHO cells and selection result in cell clones expressing VEGF-B₁₆₇ (see, e.g., Urlaub, G., and L. A. Chasin. Proc. Natl. Acad. Sci. USA (1980) 77:4216-4220). Cotransfection and selection of CHO cells is carried out using standard cell culture procedures (see, e.g., Ausubel, F. M. (ed.). CURRENT PROTOCOLS IN MOLECULAR BIOLOGY, John Wiley & Sons, Inc. 1996. and Herlitschka et al., Protein Expression and Purification (1996) 8:358-364). Screening for expression is done by Western blotting of cell culture supernatants according to methods known in the art using antibodies obtainable by immunizing rabbits with VEGF-B specific peptides (see, e.g., Towbin, H. et al., Proc. Natl. Acad. Sci. USA (1979) 76:4350-4354; see also Olofsson, B. et al., J Biol Chem (1996) 271:19310-7; these references and references cited therein are incorporated herein by reference).

A stable high expressing CHO clone is used to produce VEGF-B₁₆₇ using standard procedures in biotechnology (see, e.g., Gomperts, E. et al.,. Recombinate. Transf. Med. Rev. (1992) 6: 247-251).

Construction of the Expression Plasmid pVEGF-Bwt167.

The plasmid pVEGF-Bwt167 is constructed by insertion of a 580 bp PCR product derived from phage Lambda gt11-VEGF-Bwt167 into the expression plasmid pSI (Promega, Inc.). This phage is obtainable by screening a human fibrosarcoma cDNA library in lambda g11 (obtainable from Clontech, Inc.). The PCR reaction is performed employing the Advantage KlenTaq Polymerase Mix system (Clontech. Inc.) in a final volume of 100 microliter containing 1 ng of the plasmid template, 0.5 μM of primers P-wt167(1) 5-GATCGCTAGC GGCAGCATGA GCCCTCTGCT CCGCCGCCTG-3′ and P-wt167(2) 5′-TGACGCGGCC GCTCACCTTC GCAGCTTCCG GCACCTGCAG-3′ as well as 0.2 mM dNTPs, using the conditions 93° C. 30 sec, 55° C. 30 sec, 72° C. 30 sec for 30 cycles followed by a 72° C. 10 min extension in a Pharmacia LKB Gene ATAQ Controller PCR. system. The PCR product is gel-purified, digested with NheI and NotI and ligated into the NheI/NotI cleaved plasmid pSI. The resulting plasmid is designated pVEGF-Bwt167.

### Example 2

Coupling of Peptide GGGVFWQ to VEGF-B₁₆₇

Principle

The N-terminally blocked peptide is activated at the C-terminus by the water soluble carbodiimide EDC (N-Ethyl-N′(3-dimethylaminopropyl) carbodiimide in the presence of N-hydroxysuccinimide (NHS). The activated peptide then reacts with the primary amino groups of the VEGF molecule. By adjusting the pH carefully it is possible to direct this reaction towards the N-terminus of the VEGF-B₁₆₇ molecule (see, e.g., Staros, J. et al., Anal. Biochem. (1986) 156: 220-222 and Wong, S. S., “Application of Chemical Crosslinking to Soluble Proteins” in: CHEMISTRY OF PROTEIN CONJUGATION AND CROSSLINKING, (CRC Press Inc. 1993), pp. 221-229; these references and the references cited therein are incorporated herein by reference).

Method

1 μM of the purified peptide is dissolved in a small amount of DMSO and further diluted with buffer to give a 1 mM solution. EDC and NHS are added in a 10 fold molar excess and the reaction is allowed to take place at room temp. for 2 hours. The mixture is then transferred to a solution of the VEGF-B₁₆₇ in buffer. The pH is controlled and adjusted if necessary to 6.8. The reaction is allowed to proceed for additional 18 hours at 40° C. The separation of free peptide from VEGF-B₁₆₇/VEGF-B chimeric molecule can be performed by gel filtration. The mixture can be applied to a column filled with Sephadex G25 and the proteins can be recovered in the void volume, whereas the unreacted peptide and low molecular reaction products will be eluted later.

The purity of the VEGF chimeric molecule conjugate is assayed by standard technologies as SDS-PAGE, HPLC, N-terminal sequencing and spectrophotometry. The absolute mass of the conjugate is determined by mass spectrometry. This can provide information about the amount of coupled peptide and also on the location of the peptide on the VEGF. Ideally a molar coupling ratio is achieved where the peptide is located at the N-terminus of the VEGF. The biological activity of the conjugate is determined by appropriate animal and/or cell culture tests.

### Example 3

Coupling of a C-terminal Elongated Peptide GGGVFWQ to VEGF-B₁₆₇

Principle

To avoid sterical hindrance during the binding of VEGF-B chimeric molecule to the VEGF receptor resp. to the targeting peptide receptor the peptide can be elongated by several additional amino acids on the C-terminal end. The C-terminal spacer should allow maximal flexibility while not interfering in the binding mechanism of VEGF and/or peptide to their specific receptors. Usually poly-Gly or poly-Ala sequences fulfill these requirements.

Method

The coupling can be performed as described in Example 2, above.

### Example 4

Coupling of Peptide GGGVFWQ to VEGF-B₁₆₇ by Using a Heterobifunctional Reagent with a Spacer Domain

Principle

The coupling of the peptide can also be performed by reacting the N-terminus of the peptide with the amine-reactive part of a heterobifunctional crosslinker (for example SMBP), whereupon the activated peptide then reacts with an accessible sulfihydril group of VEGF-B₁₆₇ to form a thioether linkage (see, e.g., Staros, J. et al., Methods Enzymol. (1989) 172, 609 and Wong, S. S., “Application of Chemical Crosslinking to Soluble Proteins” in: CHEMISTRY OF PROTEIN CONJUGATION AND CROSSLINKING, (CRC Press Inc. 1993), pp. 221-229). In the case of using SMBP the length of the spacer is in the order of 1.5 nm. It has to be kept in mind that the sulfhydril group involved in the coupling reaction is not essential for the binding to the receptor protein.

Method

1 μM of the peptide containing the free N-terminus is dissolved in DMSO/buffer. A 10 fold molar excess of Sulfo-SMBP (Sulfosuccinimidyl 4-(p-maleimidophenyl)butyrate is added. After activation of the peptide for 1 hour at room temperature an equimolar amount of VEGF-B₁₆₇ is added. The coupling reaction is allowed to proceed for additional 18 hours at 40 G. The separation of the free peptide from the VEGF-B/VEGF-B chimeric molecule can be performed using gel filtration as described above.

### Example 5

Non-covalent Coupling of Peptide GGGVFWQ to VEGF-B₁₆₇

Principle

Ionic interaction is one of the dominant forces in forming protein structures. By introducing regions of opposite charge into macromolecules it is possible to form tight complexes between two reaction partners which are also stable under physiological conditions. The introduction of these charged amino acids has to be compatible with the function of both molecules.

Method

The peptide GGGVFWQ has to be modified at the N- or C-terminus by a stretch of 4-6 charged amino acids (Lysine, Arginine for the introduction of positive charges, Glutamic or Aspartic acid for the introduction of negative charges). Also the VEGF-B₁₆₇ has to be extended preferably at the N-terminus with a sequence of 4-6 charged amino acids. Once the reaction partners are synthesized and purified to the appropriate degree of quality, the complexes can be formed easily just by mixing the equivalent amounts of the opposite charged reaction partners. Separation of unreacted molecules from conjugates can be performed using Ion Exchange Chromatography.

The formation of ionic complexes can be monitored by different analytical tools. For example microcalorimetry or surface plasmon resonance can give information about stoichiometry and binding characteristics of the chimeric molecules.

Analogous to Example 3 described above, the conjugation method described in Example 4 and 5 can also be performed with elongated peptides to allow for an adequate distance between the peptide and the VEGF-B₁₆₇.

### Example 6

Conjugation of VEGF-B₁₆₇ to a His-tagged peptide GGGVFWQ

Principle

In the case a complete separation of the VEGF chimeric molecule from free VEGF-B₁₆₇ is necessary, the peptide can be elongated on the N- or C-terminal end with a stretch of 4-6 Histidine molecules. The coupling reaction is then performed according to example 2 or 5. For the capture of VEGF-B chimeric molecules, the approach of metal affinity chromatography can be used (Porath, J. et al., Nature (1975) 258: 598-599).

Method

After the coupling reaction according to example 2 is completed, the reaction mixture is passed over a column filled with a nickel-chelate gel. All molecules containing multimeric Histidines are bound to this column. After washing the column the bound proteins/peptides are eluted with a buffer containing Imidazole. The separation of the conjugate from free peptide is performed again by gel filtration as described above.

### Example 7

Coupling of Peptide CRSWNKADNRSC to VEGF-B₁₆₇

In addition to the amino and carboxyl group of the N- and C-terminus, this peptide has two functional sulfhydril groups and one -amino group of Lysine that can be used for the coupling to VEGF-B₁₆₇. If it is necessary to use the peptide in a cyclic structure, only the amino- and carboxyl groups are available. Because there are more reactive groups on the peptide, the amount of theoretical byproducts can increase.

Method

1 nM of the solubilized VEGF-B₁₆₇ is activated for 1 hour at room temperature with a 10 fold molar excess of sulfo-SMCC (Sulfosuccinimidyl 4-(N-maleimidomethyl)cyclohexane-1-carboxylate) at pH 6.8. At this pH the activation occurs preferably at the N-terminal amino group of the VEGF-B₁₆₇. The dominating side reaction will be the intramolecular crosslinking with internal free SH-groups, therefore 10 nM of the reduced peptide are added and the reaction is allowed to proceed for 18 hours at 4° C. The reaction products are purified by means of ion exchange chromatography, size exclusion chromatography or reverse phase chromatography. By using His-tagged peptides, the purification can also be performed using immobilized metal affinity chromatography. If there are antibodies available against one or both of the reaction partners the purification can also be facilitated by means of immune affinity chromatography. The same chemistry described in Examples 1 through 6 can also be used.

### Example 8

Carboxy-terminal (Ct) Fusion of the Targeting Peptides GGGVFNQ and CRSWNKADNRSC to VEGF-B₁₆₇

Construction of Plasmids pVEGF(BHG4S)₃,-GGGVFNQ and pVEGF(B)-(G4S)₃-CRSWNKADNRSC and Expression of the Chimeric Molecules in CHO Cells

The plasmids pVEGF(B)-(G4S)₃-GGGVFNQ and pVEGF(B)-(G4S)₃CRSWNKADNRSC contain the DNA sequences coding for the targeting peptides NH₂-GGGVFWQ-COOH and NH₂-CRSWNKADNRSC-COOH, respectively, fused to the C-terminus of the VEGF-B₁₆₇ molecule via a NH₂-(GGGGS) ×3-COOH hinge region. This type of linker is usually used to flexibly connect heavy and light chains in a single chain antibodies; alternatively, other connecting peptides, such as the natural hinge region present, in human immunoglobulin genes or oligo-proline or oligo-glycine linkers can be used. The linker peptide can, in addition, contain a protease cleavage site located between C-terminus of VEGF-B₁₆₇ and the linker (e.g., a plasmin cleavage site) allowing, after high affinity targeting to normal or ischemic heart, the release of a native VEGFB₁₆₇ molecule. Due to the flexibility of the linker, the C-terminal fusion peptide does not interfere with receptor binding.

A series of modular plasmids are constructed to finally obtain plasmids pVEGF(B)-(G4S)₃-GGGVFNQ and pVEGF(B)-(G4S)₃-CRSWNKADNRSC (see Materials and Methods, below). The intermediate plasmid pvegf-ss(l) provides the VEGF-B signal sequence followed by a HincII restriction site allowing for the convenient insertion of either the wild-type VEGF-B₁₆₇ sequence or any other desired N-terminal fusion peptide (see Example ‘N-Terminal fusions’). The final constructs, the plasmids pVEGF(B)-(G4S)3-GGGVFNQ and pVEGF(B)-(G4S)₃-CRSWNKADNRSC, are transfected into CHO cells. Cotransfection with a selectable marker, selection of CHO cell clones and production of the proteins can be carried out using standard cell culture and biotechnology procedures (see, e.g., Example 1). The purification of the chimeric proteins is performed according to standard protein chemistry procedures (chromatography using anion and/or cation exchange resins, gel filtration or affinity chromatography).

Materials and Methods

Construction of Plasmids

pSI-vegf-MCS(1)

In a first step the commercially available vector pSI (Promega) is cut with BglII treated with Klenow Polymerase using standard conditions and religated. The resulting intermediate plasmid is designated pSI-B. Subsequently, pSI-B is digested with NheI and NotI and ligated with annealed oligonucteotides P-vegfMCS(1) 5′-CTAGTACGTA TCTAGAGTCG ACACTAGTAG ATCTGATATC GCTAGCCTCG AGGCGGCGC CACGTGTACG TAGGCC-3′, and P-vegfMCS(2) 5′-GGCCTACGTA CACGTGGCGG CCGCCTCGAG GCTAGCGATA TCAGATCTAC TAGTGTCGAC TCTAGATACG TA-3′. The resulting plasmid is sequenced employing the primer P-4371 (5′-AATACGACTCACTATAG-3′) and designated pS1-vegf-MCS(1). pvegf-ss(1). Insertion of a DNA stretch encoding the VEGF-B₁₆₇ signal sequence Met¹-Ala²¹ including amino acid codons Pro²², Val²³ and Asp²⁷ is done by ligating the XbaI/SalI cut vector pS1-vegf-MCS(1) with the annealed oligonucleotides P-ss(1) 5′-CTAG GCCACCATGAGCC CTCTGCTCCG CCGCCTGCTG CTCGCCGCAC TCCTGCAGCT GGCCCCCGCC CAGGCCCCTG-3′ and P-ss(2) 5′-TCGACAGGGG CCTGGGCGGG GGCCAGCTGC AGGAGTGCGG CGAGCAGCAG GCGGCGGAGC AGAGGGCTCA TGGTGGC-3′. The inserted region is sequenced (primer P-4371) and the resulting plasmid 15 named pvegf-ss(1). Amino acid codons Val²³ and Asp²⁷ form a HincII restriction site. This allows for the convenient insertion of either the wildtype VEGF-B₁₆₇ sequence (codons Ser²⁴ Gln²⁵ and Pro²⁶) or for any desired N-terminal fusion peptide.

pvegf-d24/26

In order to construct the vector pvegf-d24/26, VEGF-B₁₆₇ coding sequences corresponding to amino acid residues Asp²⁷ to Arg¹⁸⁸ are amplified as a 500 bp PCR product in a standard PCR reaction employing primers 2-27/₁₆₇(1) 5′-GATCGTCGAC GCCCCTGGCC ACCAGAGGAA AGTGG-3′ and P-27/₁₆₇(2) 5′-GATCAGATCT TCGCAGCTTC CGGCACCTGC AGGTG-3′. The PCR product is digested with SalI/Bglll and the resulting 486 bp fragment is cloned into SaII/BgIll cut plasmid pvegf-ss(1).

pvegf-d24/26-dH

To delete the singular Hpal site, pvegf-d24/26 is digested with HpaI and ligated with the hexanucleotide P-AgeI(1) 5′-ACCGGT-3′ (Agel site) giving rise to the plasmid pvegf-d24/26-dH.

pVEGF(B)-F

The plasmid pvegf-d24/26.dH is digested with HincII and ligated with annealed oligonucleotides P-24/26(I) 5′-TCCCAGCCT-3′, and P-24/26(2) 5′-AGGCTGGGA-3′. The correct (sense) insertion of the oligonucleotides is confirmed by sequencing employing the primer P4371 and the resulting plasmid is designated pVEGF(B)-F. The antisense construct, having the oligonucleotides inserted in the antisense orientation is also isolated and designated pVEGF(B)-antisense.

PVEGF(B)-(G4S)₃

To complete the construction of the vector pVEGF(B)-(G4S)₃, annealed oligonucleotides P-Li(1) 5′-GATCTGGCGG CGGCGGCAGC GGCGGCGGCG GCAGCGGCGG CGGCGGCTCT G-3′, and P-Li(2) 5′-CTAGCAGAGC CGCCGCCGCC GCTGCCGCCG CCGCCGCTGC CGCCGCCGCC A-3′ encoding the (Gly-Gly-Gly-Gly-Ser) ×3 linker sequence, are inserted into BglII/NheI cut vector pVEGF(B)-F.

PVEGF(B)G4S)₃-GGGVFNQ

Construction of pVEGF(B)-(G4S)₃-GGGVFNQ is done by ligation of NheI/NotI cut vector pVEGF(B)-(G4S)₃, with annealed oligonucleotides P-D(1) 5′-CTAGC GGC GGG GGC GTG TTC TGG CAG TAAGC-3′, and P-D(2) 5′-GGCCGCTT ACTGCCAGAA CACGCCCCCG CCG-3′. The plasmid pVEGF(B)(G4S)₃,-GGGVFNQ contains the DNA sequences coding for the targeting peptide NH₂-GGGVPWQ-COOH fused to the C-terminus of the VEGF-B₁₆₇ cDNA via a NH₂-(GGGGS) ×3-COOH hinge region.

pVEGF(B)-(G4S)₃-CRSWNKADNRSC

Construction of pVEGF(B)-(G4S)₃-CRSWNKADNRSC was done by ligation of NheI/NotI cut vector pVEGF(B)-(G4S), with annealed oligonucleotides P-CRSWNKADNRSC(1) 5′-CTAGCTGCC GCAGCTGGAA CAAAGCCGAC AACCGCAGCT GCTAAGC-3′ and P-CRSWNKADNRSC(2) 5′-GGCCGCTT AGCAGCTGCG GTTGTCGGCT

### Example 9

Amino-terminal (Nt) Fusion of the Targeting Peptide CRSWNKADNRSC to VEGF-B₁₈₆

Construction of the Plasmid pVEGF(B)-Nt-CRSWNKADNRSC and Expression of the Chimeric Molecules in CHO Cells

The plasmid pVEGF(B)-Nt-CRSWNKADNRSC contains the DNA sequences coding for the heart tissue target peptide NH₂-CRS NKADNRSC-COOH inserted between the signal peptide and the N-terminus of the VEGF-B₁₈₆ molecule via a NH₂-(GGGGS) ×3-COOH hinge region. Other linker peptides containing functional elements may be used (see Example 8 above). The N-terminal fusion allows the natural proteolytic processing occurring with the VEGF-B₁₈₆ molecule without loss of the targeting molecule. Since the N-terminus appears to be located distal to the membrane binding face of the dimeric VEGF molecule, the fused targeting peptide can interact without steric hindrance with its receptor. Part of the series of modular plasmids described in example 8 is used to further construct the plasmid pVEGF(B)-Nt-CRSWNKADNRSC (see Materials and Methods). The final construct is transfected into CHO cells. Cotransfection with a selection marker, selection of CHO cell clones and production of protein is carried out using standard cell culture and biotechnology procedures (see Example 1). The purification of the chimeric proteins is done according to standard protein chemistry procedures.

Materials and Methods

Construction of Plasmids

pVEGF(B)186-d24/26

Construction of pVEGF(B)186-d24/26 is done by digestion of pvegf-d24/26-dH (see Example 8) with SalI and BglII. A 492 bp fragment is removed by gel purification. This step deletes DNA sequences coding for amino acids Asp27 to Arg188 of VEGF(B)167 from plasmid pvegf-d24/26-dH (see Example 8). Subsequently a 553 bp SalI/BglII cut PCR product coding for amino acid Asp²⁷-Ala²⁰⁷ of VEGF(B)186 is inserted. PCR is done as a standard PCR reaction employing primers P-27/167(1) and P27/186(1) (5′-TGACAGATCT CTAAGCCCCG CCCTTGGCAA CGGAGG-3′) and VEGF(B)186 cDNA as a template. In the final plasmid pVEGF(B)186-d24/26 amino acids Asp²⁷ to Arg¹⁸⁸ of VEGF(B)167 are replaced by amino acids Asp²⁷ to Ala²⁰⁷ of VEGF(B)186, amino acids Met¹ to Val²³ are common to both VEGF(B) forms whereas amino acids Ser²⁴, Gln²⁵ and Pro²⁶ are still missing.

pVEGF(B)186-Nt-R13

Construction of pVEGF(B)186-Nt-CRSWNKADNRSC is done by ligating HindII cleaved vector pVEGF(B)186-d24/26 with annealed oligonucleotides P-Nt-CRSWNKADNRSC(1) 5′-TGCCGCAGCT GGAACAAAGC CGACAACCGC AGCTGCTCCC AGCCT-3′ and P-Nt-CRSWNKADNRSC(2) 5′-AGGCTGGGAG CAGCTGCGGT TGTCGGCTTT GTTCCAGCTG CGGCA-3′. The plasmid containing the oligonucleotides inserted into the opposite direction is also isolated and designated pVEGF(B)186-Nt-antisense.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A chimeric molecule comprising an angiogenic factor linked to a targeting molecule that specifically binds to a vascular endothelium.

2. The chimeric molecule of claim 1, wherein the angiogenic factor specifically binds to at least one of VEGF-R1, VEGF-R2, or VEGF-R3.

3. The chimeric molecule of claim 1, wherein the targeting molecule is a peptide.

4. The chimeric molecule of claim 1, wherein the angiogenic factor is vascular endothelial growth factor A (VEGF-A), vascular endothelial growth factor A₁₂₁ (VEGF-A₁₂₁), vascular endothelial growth factor A₁₄₅ (VEGF-A₁₄₅), vascular endothelial growth factor A₁₆₅ (VEGF-A₁₆₅), vascular endothelial growth factor A₁₈₉ (VEGF-A₁₈₉), vascular endothelial growth factor A₂₀₆ (VEGF-A₂₀₆), vascular endothelial growth factor B (VEGF-B), vascular endothelial growth factor B₁₆₇ (VEGF-B₁₆₇), vascular endothelial growth factor B₁₈₆ (VEGF-B₁₈₆), vascular endothelial growth factor C (VEGF-C), vascular endothelial growth factor D (VEGF-D), vascular endothelial growth factor E (VEGF-E), placental growth factor (PlGF), acidic fibroblast growth factor (aFGF), basic fibroblast growth factor (bFGF), or angiopoietin-1 (Ang1).

5. The chimeric molecule of claim 1, wherein the angiogenic factor is Ang2, endostatin or angiostatin.

6. The chimeric molecule of claim 1 that is a fusion protein, wherein the fusion protein comprises an angiogenic factor linked to a targeting molecule that specifically binds to a vascular endothelium.

7. The fusion protein of claim 6, wherein the angiogenic factor is VEGF-B, vascular endothelial growth factor B₁₆₇ (VEGF-B₁₆₇), vascular endothelial growth factor B₁₈₆ (VEGF-B₁₈₆), or vascular endothelial growth factor C (VEGF-C).

15. A method of increasing cardiac neovascularization comprising contacting endothelial cells of the cardiac vasculature with a chimeric molecule wherein the chimeric molecule comprises an angiogenic factor linked to a targeting molecule that specifically binds to a vascular endothelium.

16. The method of claim 15, wherein the angiogenic factor specifically binds to at least one of VEGF-R1, VEGF-R2, or VEGF-R3.

17. The chimeric molecule of claim 15, wherein the targeting molecule is a peptide.

18. The method of claim 15, wherein the angiogenic is vascular growth factor A (VEGF-A), vascular endothelial growth factor A₁₂₁ (VEGF-A₁₂₁), vascular endothelial growth factor A₁₄₅ (VEGF-A₁₄₅), vascular endothelial growth factor A₁₆₅ (VEGF-A₁₆₅), vascular endothelial growth factor A₁₈₉ (VEGF-A₁₈₉), vascular endothelial growth factor A₂₀₆ (VEGF-A₂₀₆), vascular endothelial growth factor B (VEGF-B), vascular endothelial growth factor B₁₆₇ (VEGF-B₁₆₇), vascular endothelial growth factor B₁₆₇ (VEGF-B₁₈₆), vascular endothelial growth factor C (VEGF-C), vascular endothelial growth factor D (VEGF-D), vascular endothelial growth factor E (VEGF-E), placental growth factor (PlGF), acidic fibroblast growth factor (aFGF), basic fibroblast growth factor (bFGF), or angiopoietin-1 (Ang1).

19. The method of claim 15, wherein the chimeric molecule is a fusion protein wherein the fusion protein comprises an angiogenic factor linked to a targeting molecule that specifically binds to a vascular endothelium.

20. The method of claim 19, wherein the angiogenic factor is vascular endothelial growth factor B, vascular endothelial growth factor B₁₆₇ (VEGF-B₁₆₇), vascular endothelial growth factor B₁₈₆ (VEGF-B₁₈₆), or vascular endothelial growth factor C (VEGF-C).

21. The method of claim 15, wherein the chimeric molecule is suspended or dissolved in a pharmaceutically acceptable carrier.

22. The method of claim 15, wherein the chimeric molecule is suspended or dissolved in a cell culture medium.

23. The method of claim 15, wherein the pharmaceutical composition is in the form of an injectable solution.

24. A polynucleotide comprising a nucleic acid sequence encoding a fusion protein comprising an angiogenic factor and a targeting molecule, wherein the targeting molecule specifically binds to a vascular endothelium.

25. The polynucleotide of claim 24, wherein the nucleic acid sequence is in an expression cassette.

26. The polynucleotide of claim 25, wherein the expression cassette is in a retroviral vector or an adenovirus-associated vector.

27. A method of inducing angiogenesis in a tissue comprising transfecting an endothelial cell with the nucleic acid of claim 24, whereby the cell expresses a fusion protein encoded by the nucleic acid.

28. A pharmaceutical composition comprising the chimeric molecule of claim 1 and a pharmaceutically acceptable carrier.

29. A pharmaceutical composition comprising the fusion protein of claim 6.
